SEQUENCE LISTING (18 sequences)

SEQ ID NO: 1 - protein, 116 aa, Homo sapiens
  VARIANT (1)...(116): precursor
MKGLRSLAATTLALFLVFVFLGNSSCAPQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 2 - protein, 90 aa, Homo sapiens
  PEPTIDE (1)...(90): mature peptide
APQRLLERRNWTPQAMLYLKGAQGRRFISDQSRRKDLSDRPLPERRSPNPQLLTIPEAATILLASLQKSPEDEEKNFDQTRFLEDSLLNW

SEQ ID NO: 3 - DNA, 8901 bp, Homo sapiens
  misc_feature (1)...(8901): public sequence AC006559 (155800..164700)
agagattgtttattcttggctttaatttatcatatcatattcactattcaaccaactccaatcttgcttctatcctcaaaaccatcatattccaaatccagtggatgtttcttggagctaatgttgcttgacctcccaatactactggatcttcacttggcttccatgatatcagggtcccctggaatttctcctttctttctgactctacctcaacctccttttctggctcttcctctgctcattctctatgcgtttgttttcatagctctgtccctgccactcttctttagccctattcactccctaagtgttgtgtttaatctcatccctttactggttgtaaaattcttagaattcttaaaattcttaaagctaatcatttctgccccccaccaaaaagaaaatctgtaggctggtgacctcttggctgacctctatcaagttttgcatttctaaccttctattttgtacttgtacttggatatttttaatatacattttaattttaatatgcctatatggcaagtgttaatttccctctcataacttgcttcttaagaagtttctctaatttcagtaaattacttcataatttacccacagttttatccataaacctaagaatcatccttgatttatttttctcacactttcatatcccttttatatacaagttctatattttctacctccacaacatgtattccaaattcatttacagctcttctttccctgtgtgtaccatcccagtgtaagccatcatcatctattgcttataatcgtatttccacatcaactggcccatttcttcttctccaatctcatttcaaagcacctcccccgttcacccacacaacgctgcctccattggcctctttcctagtccccaagcaagtcatgctctttgctgtccacagattttgcttttctttcttctattgagaatgcctggaatactaattcctgcccagcttctcccattacttcaccttatccttcaagactaccctctgtttttaaccttccctaaagtagcccaccacttatcctatcacatcaccttgcttacttttacttaacaatttaaaacattctgtagttacagtagtccctgcttatccacggtttcactttccatggtttcagttacccatggtcaactacagtctgaaaatattaaatgggaaattccagaattaataagtttaaattgtgagctttaaatctgagtagggtgttgaaatctctcactgtccagctccagcccacccaagacatgaaccatctttttgtccagcatgtccatatgtaaacaccactctcccattaatcagttagtaaccatcttggtgaacagataaaaaaaaatagtataataaggcatatacgtatcagtggtttcaggcatccactgtgggtcttgaaacatgctcctccaccccacacacacctacgattaaggggtaactactgtgtcttatttatctattcatcagtttactgtgtaccttaactagtaggtaacttagatcaaagcagagaccatgtcttttcagtgcatggctgtgtcccttgaaatacaaggattgtgtgtctcataggcactcaataaatatttgctaaacaacagaatgaattgataattaagtgaatgactgatgtcaaattccattgagattcccaaaccttttcatacatctttagccatcagtgatagactgatggctagttacccaagaaaagttttctgatttattcttttacttttgattctgccttggactattgattgttttcattaataataataataacatgttactttgggcagctagtaggaaaagcaaggccaatgatgctgacacattctgaaggcctcctgggtgatgcaatcagtatttaaacaaataatttctgcccatcaatcattcctctacttcctgcaatgaagctccctggaggagtcatcagattctcaatcttgcttgaagactgacaagatgtccctgtggactcccaaactctactccagatggggaggtgcccttaacaccaagattttaaaagctccaatttcagagcaagagtcgaaaactcacagataaagttatagttatttcagggttctgaaaagacgcagaacatgaaggtaagtaaaggctttattaacttgagacttcttagctattttttaaaacgttttatagcattttacctatttcatgtttaataaggattttagagtttaaaggatatttgaacgatacgcctttagaataacagaatcatttgattttactaagaaaaaaattcacgtatttacttattcttcatttgggtgaagtaatacaaataatatgtttctaaatacgttttttcaagtggactctttgtgaattaactagaaggtagaagggaaacacctccagaaaactgagtattaaaacagaatattgcgagaagtaagggggtttataattgtccaggcgtcaaggaaggaagcagaagatcctattggcacagaaatgaccctttctggttgatcgcttcatatagggactcagaagtctggcagcaacaaccttggctcttttcctggtgtttgttttcctgggaaactccagctgcgctccgcaggtaatcaaatgcaaaataaaaaattttaaaacaatgcgcactgtgtgtccactctgctctttctttcttccttcttgttttattctcttctttttcctttattttgctgtaatagaggactgagctgcaatgttttattaactgctcttcccttcccccgggctatagagactgttggagagaaggaactggactcctcaagctatgctctacctgaaaggggcacgtaagttccaaatatttcgctcttcctacaataatggaagacccctaggagtcaggaacagatagatggagagttatggagagagaataatgtcgagtttattcccttaaatcatcgaggccatcaaagagaccggtaggtgagggaggggtgggagaggggaagagtccctgattattggaaatcctccatccttattagaaattctccatccaaaactggagggttgcttctcttggtccaaatggggactcgggttgcctgggaaatatctcagtaacccgacctccgctccaaagcgcccttgcggcgtccggtcaggcgcggggctttcctccagagctccagggcccctggctcagcccggggttgcgctgggagcgctggaaactcggcagccccgcgcgaccctatcctggagcaacctgccccctccccacgcccggggactgcgctgtgccgggaggagctgaggtttaagcccgggttgtcccgggccaggctgtcgctgagccccaggtctcgtttttgcagagggtcgccgcttcatctccgaccagagccggagaaaggacctctccgaccggccactgccgggtgagtgaccaagggtgcaagggcgctagtcctgcgcttttggaataggatggggcgggcagggcttgctccgcgctggtgccgaaagaaagcgtctcctcaccggaaagacgcggctctgaggcgccctcagatacagtccgcccgagggcagcccgggttggcagcagcagcagctggatgccgagcgccggagctgggagctcgcgcgtccagccccgcgccaatggtggcaagggcggcccaggctggcgctgcggctccccaagccttattggcttgcggctgttcagccagccctctggctgccagggtgtgcagaggtccccagggagttaggacctgaggtgcagctcagaggggtaaggcgagataggaagaactatgcaaatgcttctagaaatgtatctgcgcgtttgtgcaggaactttactcagcctcctcttaaacgctgttattattaaaataagaacttacctaggaacttcaaatgtatgcttttgtttgactagaaagaactatcaaaggttcaaaatcaaaagagtaacatgcgggcagttaaggatgggaccggggactgttctttttcatgataaacctttccatcgtattgtattaacaaataacaatacaatttgatgaaattgtaaagattgaaatgctaaaattaaaaagtatacttacttggaaaaataccaaacactgttgaacacatagtaggtgcttaaatttttgtttaggctcttcaaacatgctgtgtaattgagcttatttattttatgaggttaagatttgtttctcaacttcagttgccccaaattagaaatcatgttttgtgctgaatcagaacaatttgtcttttttcaacttagatttgaaataactttgagttcttagcagatactccttcagtcaaataattaccttatatttggccgcaaaggttaaattattatctaaaattctctttggcatatagtttaacattctcttccataccctgtttgtaaacttgacataaccaattgtaagtcatttccaaggcaattgtgacatgttaaaaaattagtcttctgtgtaagtagtcctttgttgaggggtcaaaatctatttcatagaggtttttctagtattgctgcatgttagaataggacttatctacatcaatatataaatgctaagagaatctgtatacatttttgtttctgcagaaagacgaagcccaaatccccaactactaactattccggaggcagcaaccatcttactggcgtcccttcagaaatcaccagaaggtactagcatagtggcctctttcacctgcataacagaacagctttgcttactttcgggattctgtgttgagtgcaacacccctcccccagaatttcttggccttaggcccagaaattctaattctgtactgcttgagtgaggcccaagaatctctgtccttaagaagttcttccacattattctgatgattggtcaggtttgaaaacaggaacttcagagcccgttaataaaggttgaaatcatgagaatggagataccttctagggcttagatatagtgggaaaagggaagacaataaaacacccaaaaatcaagggaaatggggggaaatagtaccagtgaagggacattaaaaggagcaagcagaaaggtaagaggaaaatcagaataagagagtatcccagaattcattcagtagctgatgctactgatcccgctcctcttgaaactttccagggatggaaactatcagaatggttactatagcagaatggttagtaagaatacagaactcaggggacattaaacagtgtgtcacaggacttccccccacctcctgcccagtgacacagcttgctgctgctattccatatctgaaactttgtttacagaatgtgatacaagaaagtaaactgcaaagttaagagaaagcaggtctcaggctctggagagctggcatgactctcagaactcttgcagtgaaaaggtgtgaaatagttgaataacgggtagaggatgctgtatccaaggtgttctttttaaaacccaagacatggaacccagcgatttttggagtcagtacgaaaagagctctgatggagattggttgatagtgacgttttagatttgattgttgggaaaatgtgcagagccagaaaggaaagtgacttataatggacaagagagagctaattttaaagaggagaggtatcttcattagcaatttgaggaaaggatgtgattataggtccccatctcaagtcttttttggaaaataaatatgtatttttttaattattgaagttttctaagtggattgtctctgaagatgaaatgactaattttattagtctaaattgatagaaaagtttttgattcccatatgataaattaatcactagtcttctttagtatttttattctatactacctcattattatttagaaggctcttatttagtgaattatggtttcagtggaatttatacagggacgccgttctggttttgttttgttttgttttgttttcttccttccctttaaagagaagcccattttctgcaatggtttaaagtgtgctagaaattcaggttaagatgtaacccaaacattcaaattatattgcaactaactgaaagaatgtgcttttatctagaggtgatcttagaagaaattagtagatgcactttgcccctggccactgcttctccctaccgtattgtaacttttagtcattctatcctatttttagagtcccaggaacctgctattgtctaactctttaagtatccccacctcagaagtgatacctcatgctctaaagtttcgcgatcccagccagcaaacgagtgcctcttctttttgtctcatttcctccccagaactctagctcccctaataaaagaacatttgcaaatgtgttcccatgtcatctttcccgagcaagatccattttaacaggaaatataaagagacagtcctttgatgcaagtgaattattttaatggtgggtattttagatataagtcaatctgtgagagcaagacacatgacttttgagatttttaccagacataccatactgtagtttcagtcattatagaggttcagatgtaaaatatgaattccccaatgtgtcccaagctgagaaaaaaagattttaaactgagcaaatcaaagcccatattgtttaaattttcagtagcagagtccataagttaacatagcttagaaggctaaatgtttatttcctgagattctctttgtcttaccttttcctctattaaaacgcagagtgtatatttattttaaagaatttttttttcttacagatgaagaaaaaaactttgatcaaaccagattcctggaagacagtctgcttaactggtgaaaatatactggattatgtttaattatggttctattctctttgaaaacatgaaccatgtgaataaaacctttggacccttttattccatttgtaatcttaagaacacacacagatagttttattctttcagaaacaaaatatatataggatgcttagctgagaacatcatcttctttcattgcttcaggtcctgtttagatgaccaaaaatgttttcagatcaccttgtgtcttactcttgagtttcttagaatatttataattataaggctgaagactaaagtgttctttccttttaactatagccagtacctgtcttgatcttagttgtgtttttttttcattttgttacccacttgcattttgttttcactcagcagaaattctccttctctgttttccttttatcccatccccaagaatgtggaaggaaggtgagaaacatggcaggatgggaaataggagagtatgactctctatagctcatccaggagtaatcaattaagaagataaattggatgactgtggagaagctctgtgataggaacacttcagtgtggttgctgagaggagacagtcattgaggtagaaggtttgccaaagatccagagctcagagctccctttgtgctctttgggaattaccttgcattcagtttagaaacatggatctaaaagttactgggaaataagcagatggagacacactctgttgtttacgtattggaagaagggaacaagccagttttgttagaggtaactcattttccatgaccaaacagactcaacagattcaagtactctgcttactctaattgactagactctaggttttatttgacatcatagcattacataaatcactctgataacataagtgcacagtaatatgcctgatctcttcctttttaaaagccaacttgagttcagtaccatctgaatacacacacatgcacatatacccacacacgcatacacacatactcctgtggcaaacataataatgtatttatttagaattataatatgaccatcatgttaattattttttacctaatcagagttgttattgacaaatgtcataagtggaaagtattaattcttattgtcatcagtatttagccattatttagtagctcaagaatatctttatgtgaatgtctctgtaacttggaattgcaatttcactgtgttaagtaatcagaactctgcttataagatttatctgtatcttgtttcataatttaataatgaaactaaattcaagttaatgtaatgttgatctccgtcgaaaaataacttgtgagcattaaaatatctgtatggcattataataggcacctgtcattaattatgatttgatttgaaagtagacttactaggtagtgcttacatctgatttattgaaaattttttccaaaagcattgtcttttatttatacagtcctaaccacttcaccattaggaggggtgggacattaaaaattatttacccatcagacataaaattgtacaaattgactattcaaaatagtatgtgtattcaaagaacacttttaacatctatcttatgaaataattcttcctacaactaaccagaacaatctcaaaaatgtcatactaaccattgttttaaaagctgtctcaatgatcaatagctgttaaaatatagctccaagtattctaagtactcaaattccttgaatgttcttatgtacaaatatgctgattaattaaaaggtccctcttcccaattttctataactttccaactctcttatggtgtctatattctgtttatttctagatttttaaaaaaccaaaatgaacaagtgatcatagatcagaaaaccttcacattatatggcttgtgttttaaaattgggaattttctggcttcaacacacagatacatgcactagaataccttgatctgcagggagcgctttcagccactttctgcacagttctcattgcccttctcttttttgcctctatagcctacctcggctatctggcctccatcctgggctc

SEQ ID NO: 4 - DNA, 157 bp, Homo sapiens
  misc_feature (1)...(157): exon 1
ctgacaagatgtccctgtggactcccaaactctactccagatggggaggtgcccttaacaccaagattttaaaagctccaatttcagagcaagagtcgaaaactcacagataaagttatagttatttcagggttctgaaaagacgcagaacatgaag

SEQ ID NO: 5 - DNA, 81 bp, Homo sapiens
  misc_feature (1)...(82): exon 2
ggactcagaagtctggcagcaacaaccttggctcttttcctggtgtttgttttcctgggaaactccagctgcgctccgcag

SEQ ID NO: 6 - DNA, 58 bp, Homo sapiens
  misc_feature (1)...(58): exon 3
agactgttggagagaaggaactggactcctcaagctatgctctacctgaaaggggcac

SEQ ID NO: 7 - DNA, 63 bp, Homo sapiens
  misc_feature (1)...(63): exon 4
agggtcgccgcttcatctccgaccagagccggagaaaggacctctccgaccggccactgccgg

SEQ ID NO: 8 - DNA, 84 bp, Homo sapiens
  misc_feature (1)...(84): exon 5
aaagacgaagcccaaatccccaactactaactattccggaggcagcaaccatcttactggcgtcccttcagaaatcaccagaag

SEQ ID NO: 9 - DNA, 140 bp, Homo sapiens
  misc_feature (1)...(140): exon 6
atgaagaaaaaaactttgatcaaaccagattcctggaagacagtctgcttaactggtgaaaatatactggattatgtttaattatggttctattctctttgaaaacatgaaccatgtgaataaaacctttggaccctttt

SEQ ID NO: 10 - DNA, 639 bp, Homo sapiens
  misc_feature (1)...(639): public sequence BC004336
ctgacaagatgtccctgtggactcccaaactctactccagatggggaggtgcccttaacaccaagattttaaaagctccaatttcagagcaagagtcgaaaactcacagataaagttatagttatttcagggttctgaaaagacgcagaacatgaagggactcagaagtctggcagcaacaaccttggctcttttcctggtgtttgttttcctgggaaactccagctgcgctccgcagagactgttggagagaaggaactggactcctcaagctatgctctacctgaaaggggcacagggtcgccgcttcatctccgaccagagccggagaaaggacctctccgaccggccactgccggaaagacgaagcccaaatccccaactactaactattccggaggcagcaaccatcttactggcgtcccttcagaaatcaccagaagatgaagaaaaaaactttgatcaaaccagattcctggaagacagtctgcttaactggtgaaaatatactggattatgtttaattatggttctattctctttgaaaacatgaaccatgtgaataaaacctttggaccctttttaaaaaaaaaaaaaaaaaaaaaaaaaaaaaaaaaaaaaaaaaaaaaaaaaaaaaaa

SEQ ID NO: 11 - protein, 9 aa, Homo sapiens
  PEPTIDE (1)...(9): predicted active peptide 1
APQRLLERR

SEQ ID NO: 12 - protein, 17 aa, Homo sapiens
  PEPTIDE (1)...(17): predicted active peptide 2
NWTPQAMLYLKGAQGRR

SEQ ID NO: 13 - protein, 14 aa, Homo sapiens
  PEPTIDE (1)...(14): predicted active peptide 3
NWTPQAMLYLKGAQ

SEQ ID NO: 14 - protein, 8 aa, Homo sapiens
  PEPTIDE (1)...(8): predicted active peptide 4
FISDQSRR

SEQ ID NO: 15 - protein, 9 aa, Homo sapiens
  PEPTIDE (1)...(9): predicted active peptide 5
FISDQSRRK

SEQ ID NO: 16 - protein, 11 aa, Homo sapiens
  PEPTIDE (1)...(11): predicted active peptide 6
DLSDRPLPERR

SEQ ID NO: 17 - protein, 12 aa, Homo sapiens
  PEPTIDE (1)...(12): predicted active peptide 7
KDLSDRPLPERR

SEQ ID NO: 18 - protein, 44 aa, Homo sapiens
  PEPTIDE (1)...(44): predicted active peptide 8
SPNPQLLTIPEAATILLASLQKSPEDEEKNFDQTRFLEDSLLNW

(19) United States
(12) Patent Application Publication
(10) Pub. No.: US 2004/0234537 A1
(43) Pub. Date: Nov. 25, 2004

(54) Carcinoma-related peptides

(76) Inventors: Lydie Bougueleret, Petit-Lancy (CH); Anne Niknejad, Bellevue (CH)

Correspondence Address: PERKINS COIE LLP, P.O. BOX 2168, MENLO PARK, CA, 94026, US

(21) Appl. No.: 10/482,985

(22) PCT Filed: Jul. 5, 2002
(86) PCT No.: PCT/EP02/07542

Related U.S. Application Data
(60) Provisional application No. 60/303,441, filed on Jul. 6, 2001.

(51) Int. Cl. G01N 33/574; C07H 21/04; A61K 39/00; C07K 14/47

(52) U.S. Cl. 424/185.1; 435/7.23; 435/69.3; 435/320.1; 435/325; 530/350; 536/23.5

(57) ABSTRACT

The present invention is directed to methods and compositions for detecting the presence of or for measuring the levels of one or more carcinoma-related peptides in biological samples, especially human tissues. The invention further includes methods and kits for monitoring the levels of carcinoma-related peptides in an individual.

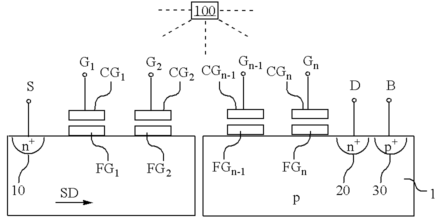

### FIELD OF THE INVENTION

The invention relates generally to human peptides found in carcinomas and other neoplasia, and particularly, to the use of such peptides as tissue and disease markers.

### BACKGROUND

The ability to detect and diagnose cancer through the identification of tumor markers is an area of widespread interest. Tumor markers are substances, typically proteins, glycoproteins, polysaccharides, and the like, which are produced by tumor cells and characteristic thereof. Often, a tumor marker is produced by normal cells as well as tumor cells. In the tumor cell, however, the production is in some way atypical. For example, production of the tumor marker may be greatly increased in the cancer cell. Alternatively, proteins and other substances normally present within or on the normal cells may be released or shed into circulation when the cell becomes malignant. Detection of surface of such secreted substances in serum may thus be diagnostic of the malignancy.

Another problem encountered in cancer diagnosis relates to identifying the cellular origin of both primary and metastatic tumors. With both types of tumors, it is sometimes difficult to morphologically distinguish among tumors of differing cellular origins, e.g., tumors of epithelial origin (carcinomas), tumors originating in the non-epithelial connective tissue (sarcomas) and lymphoid tumors (lymphomas) Moreover, with breast epithelial cancers, it is often difficult to distinguish among tumors originating in ductal epithelial tissues, secretory epithelial tissues, and myoepithelial tissues.

Therefore, it is desirable to identify previousy unrecognized tumor markers, particularly tumor markers which are secreted into circulation and which may be identified by serum assays. It is also desirable to develop methods and compositions which allow determination of the cellular origin of a particular tumor

### SUMMARY OF THE INVENTION

The present invention is directed to methods and compositions for detecting the presence and/or measuring the levels of peptide tumor markers (referred to herein as “carcinoma-related peptides”) in biological samples, especially human tissues. The invention further includes methods and kits for monitoring the levels of the carcinoma-related peptides in an individual.

As used herein, the term “carcinoma-related peptides” refers to the peptides having amino acid sequences of SEQ ID NO: 11 through SEQ ID NO: 18.

In one aspect, the invention includes the carcinoma-related peptides, which are useful in constructing assays for detecting their presence or measuring their levels in biological samples. More particularly, carinoma-related peptide of the invention is a peptide having an amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

In another aspect, the invention includes kits containing at least one monoclonal antibodies, each such monoclonal antibody being specific for a different peptide having amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

### DEFINITIONS

The terms “peptide” or “peptide” or “peptide fragment” as used herein refers to a compound made up of a single unbranched chain of amino acid residues linked by peptide bonds. The number of amino acid residues in such compounds varies widely; however, preferably, peptides referred to herein usually have from six to forty amino acid residues. peptides and peptide fragments referred to herein usually have from a few tens of amino acid residues, e.g. 20, to up to a few hundred amino acid residues, e.g. 200, or more. Generally, peptides are manufactured more conveniently by recombinant DNA methods.

The term “protein” as used herein may be used synonymously with the term “peptide” or may refer to, in addition, a complex of two or more peptides which may be linked by bonds other than peptide bonds, for example, such peptides making up the protein may be linked by disulfide bonds. The term “protein” may also comprehend a family of peptides having identical amino acid sequences but different post-translational modifications, such as phosphorylations, acylations, glycosylations, and the like, particularly as may be added when such proteins are expressed in eukaryotic hosts.

Amino acid residues are referred to herein by their standard single-letter or three-letter notations: A, alanine; C, cysteine; D, aspartic acid; E, glutamic acid; F, phenylalanine; G, glycine; H, histidine; I, Isoleucine; K, lysine; L, leucine; M, methionine; N, asparagine; P, proline; Q, glutamine; R, arginine; S, serine; T, threonine; V, valine; W, tryptophan; Y, tyrosine.

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. In reference to a triplex, the term means that the triplex consists of a perfectly matched duplex and a third strand in which every nucleotide undergoes Hoogsteen or reverse Hoogsteen association with a basepair of the perfectly matched duplex. Conversely, a “mismatch” in a duplex between a tag and an oligonucleotide means that a pair or triplet of nucleotides in the duplex or triplex fails to undergo Watson-Crick and/or Hoogsteen and/or reverse Hoogsteen bonding.

The term “percent identical,” or like term, used in respect of the comparison of a reference sequence and another sequence (i.e. a “candidate” sequence) means that in an optimal alignment between the two sequences, the candidate sequence is identical to the reference sequence in a number of subunit positions equivalent to the indicated percentage, the subunits being nucleotides for polynucleotide comparisons or amino acids for peptide comparisons. As used herein, an “optimal alignment” of sequences being compared is one that maximizes matches between subunits and minimizes the number of gaps employed in constructing an alignment. Percent identities may be determined with commercially available implementations of algorithms described by Needleman and Wunsch, J. Mol. Biol., 48: 443-453 (1970)(“GAP” program of Wisconsin Sequence Analysis Package, Genetics Computer Group, Madison, Wis.). Other software packages in the art for constructing alignments and calculating percentage identity or other measures of similarity include the “BestFit” program, based on the algorithm of Smith and Waterman, Advances in Applied Mathematics, 2: 482-489 (1981) (Wisconsin Sequence Analysis Package, Genetics Computer Group, Madison, Wis.). In other words, for example, to obtain a peptide having an amino acid sequence at least 95 percent identical to a reference amino acid sequence, up to five percent of the amino acid residues in the reference sequence many be deleted or substituted with another amino acid, or a number of amino acids up to five percent of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence of in one or more contiguous groups with in the references sequence. It is understood that in making comparisons with reference sequences of the invention that candidate sequence may be a component or segment of a larger peptide or polynucleotide and that such comparisons for the purpose computing percentage identity is to be carried out with respect to the relevant component or segment.

The term “isolated” in reference to a peptide or polynucleotide of the invention means substantially separated from the components of its natural environment. Preferably, an isolated peptide or polynucleotide is a composition that consists of at least eighty percent of the peptide or polynucleotide identified by sequence on a weight basis as compared to components of its natural environment; more preferably, such composition consists of at least ninety-five percent of the peptide or polynucleotide identified by sequence on a weight basis as compared to components of its natural environment; and still more preferably, such composition consists of at least ninety-nine percent of the peptide or polynucleotide identified by sequence on a weight basis as compared to components of its natural environment. Most preferably, an isolated peptide or polynucleotide is a homogeneous composition that can be resolved as a single spot after conventional separation by two-dimensional gel electrophoresis based on molecular weight and isoelectric point. Protocols for such analysis by conventional two-dimensional gel electrophoresis are well known to one of ordinary skill in the art, e.g. Hames and Rickwood, Editors, Gel Electrophoresis of Proteins: A Practical Approach (IRL Press, Oxford, 1981); Scopes, Protein Purification (Springer-Verlag, New York, 1982); Rabilloud, Editor, Proteome Research: Two-Dimensional Gel Electrophoresis and Identification Methods (Springer-Verlag, Berlin, 2000).

The term “oligonucleotide” as used herein means linear oligomers of natural or modified monomers or linkages, including deoxyribonucleosides, ribonucleosides, anomeric forms thereof, peptide nucleic acids (PNAs), and the like, capable of specifically binding to a polynucleotide by way of a regular pattern of monomer-to-monomer interactions, such as Watson-Crick type of base pairing, base stacking, Hoogsteen or reverse Hoogsteen types of base pairing, or the like. Usually, monomers are linked by phosphodiester bonds, or analogs thereof, to form oligonucleotides ranging in size from a few monomeric units, e.g. 3-4, to several tens of monomeric units, e.g. 40-60. Whenever an oligonucleotide or polynucleotide is represented by a sequence of letters, such as “ATGCCTG,” or the lower case equivalent, it will be understood that the nucleotides are in 5′□3′ order from left to right and that “A” denotes deoxyadenosine, “C” denotes deoxycytidine, “G” denotes deoxyguanosine, “T” denotes thymidine, and “U” denotes uridine, unless otherwise noted or understood for their context. Usually oligonucleotides of the invention comprise the four natural nucleotides, and they are joined to one another by natural phosphodiester linkages; however, they may also comprise non-natural nucleotide analogs and may also contain non-natural inter-nucleosidic linkages, particularly when employed as antisense or diagnostic compositions. It is clear to those skilled in the art when oligonucleotides having natural or non-natural nucleotides may be employed in accordance with the invention, e.g. where processing by enzymes is called for, usually oligonucleotides consisting of natural nucleotides are required.

As used herein, “nucleoside” includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the only proviso that they are capable of specific hybridization. Such analogs include synthetic nucleosides designed to enhance binding properties, reduce complexity, increase specificity, and the like.

As used herein, “detectable duplex” in reference to a hybridization assay means that for any type of signal generating means used the signal-to-noise ratio is at least two. Preferably, such signal-to-noise ratio is at least three, and more preferably, such signal-to-noise ratio is at least five,

As used herein, the term “alternative splicing” in reference to a gene means different exon combinations are included in transcripts from the same gene during RNA processing. As used herein, the term “splice variant” refers to a peptide or protein encoded by a transcript which resulted from alternative splicing.

### DETAILED DESCRIPTION OF THE INVENTION

The invention relates to methods and compositions for detecting the presence of or for measuring the amounts of one or more carcinoma-related peptides in a biological sample. In particular, the invention includes kits for performing quantitative immunoassays to detect the abundances of carcinoma-related peptides. Preferably, monoclonal antibody kits of the invention include at one monoclonal antibody, each being specific for one of the carcinoma-related peptides and not cross-reactive with the others. Kits of the invention further include labeling means to detect the quantity of each monoclonal antibody bound to its target protein. Preferably, quantitative measurements are made using a conventional ELISA format, as described more fully below.

Below procedures are disclosed for synthesizing the carcinoma-related peptides and for using the synthesized peptides to manufacture monoclonal or peptide antibodies.

The antibodies of the present invention find use in diagnostic assays for the determination of carcinoma-related peptides. The antibodies of the invention may be used in most assays involving antigen-antibody reactions. The assays may be homogeneous or heterogeneous. In a homogeneous assay approach, the sample can be a biological sample or fluid such as serum, urine, whole blood, lymphatic fluid, plasma, saliva, and the like, cells, tissue, and material secreted by cells or tissues cultured in vitro. The sample can be pretreated if necessary to remove unwanted materials. The immunological reaction usually involves the specific antibody, labeled analyte, and the sample suspected of containing the analyte. The analyte can be directly labeled with the label or indirectly labeled with a means for incorporating the label such as conjugation of the analyte to biotin and having labeled avidin or anti-biotin. The signal from the label is modified, directly or indirectly, upon the binding of the antibody of the labeled analyte. Both the immunological reaction and detection of the extent thereof are carried out in a homogeneous solution. Labels which may be employed as part of a signal producing system capable of producing a signal in relation to the amount of analyte in the sample include free radicals, chromogens, such as fluorescent dyes, chemiluminescers, enzymes, bacteriophages, coenzymes particulate labels and so forth.

In a heterogeneous assay approach, the reagents are usually the sample, the specific antibody, and means for producing a detectable signal. The specimen is generally placed on a support, such as a plate or a slide, and contacted with the antibody in a liquid phase. The support is then separated from the liquid phase and either the support phase or the liquid phase is examined for a detectable signal employing means for producing such signal or signal producing system. The signal is related to the presence of the analyte in the sample. Means for producing a detectable signal includes the use of radioactive labels, fluorescers, enzymes, and so forth. Exempary of heterogeneous immunoassays are the radioimmunoassay, immunofluorescence methods, enzyme-linked immunoassays, and the like.

One embodiment of an assay employing an antibody of the present invention involves the use of a surface to which the monoclonal antibody of the invention is attached. The underlying structure of the surface may take different forms, have different compositions and may be a mixture of compositions or laminates or comthereof. The surface may assume a variety of shapes and -forms and may have varied dimensions, depending on the manner of use and measurement. Illustrative surfaces may be pads, beads, discs, or strips which may be flat, concave or convex. Thickness is not critical, generally being from about 0.1 to 2 mm thick and of any convenient diameter or other dimensions. The surface typically will be supported on a rod, tube, capillary, fiber, strip, disc, plate, cuvette and the like. The surface will typically be porous and polyfunctional or capable of being polyfunctionalized so as to permit covalent binding of the monoclonal antibody of the invention as well as to permit bonding of other compounds which form a part of a means for producing a detectable signal. A wide variety of organic and inorganic polymers, both natural and synthetic, and combinations thereof, may be employed as the material for the solid surface. Illustrative polymers include polyethylene, polypropylene, poly(4-methylbutene), polystyrene, polymethracrylate, poly(ethylene terephthalate), rayon, nylon, poly(vinyl butyrate), silicones, polyformaldehyde, cellulose, cellulose acetate, nitrocellulose, latex, etc. Other which may be employed include paper, glasses, ceramics, metals, metaloids, semiconductor materials, cermets, silicates or the like. Also included substrates that form gels, gelatins, lipopolysaccharides, silicates, agarose and polyacrylamides or polymers which form several aqeuous phases such as dextrans, polyalkylene glycols (alkylene of 2 to 3 carbon atoms) or surfactants such as phospholipids. The binding of the monoclonal antibody of the invention to the surface may be accomplished by well known. techniques, commonly available in the literature. See, for example, “Immobilized Enzymes,” Ichiro Chibata, Press, New York (1978) and Cuatrecasas, J. Bio. Chem., 245: 3059 (1970). In carrying out the assay in accordance with this of the invention the sample is mixed with aqueous medium and the medium is contacted with the surface having a monoclonal antibody of the invention bound thereto. Members of a signal producing system and any ancillary materials may also be included in the aqueous medium, either concurrently or added subsequently so as to provide a detectable signal associated with the surface. The means for producing the detectable signal can involve the incorporation in the of a labeled analyte or it may involve the use of a second monoclonal antibody having a label conjugated thereto. Separation and washing steps will be carried out as needed. The signal detected is related to the presence of Carcinoma-related peptide in the sample. It is within the of the present invention to include a calibration as the measurement surface on the same support. A particular embodiment of an assay in accordance with the present invention, by way of illustration and not limitation, involves the use of a support such as a slide or a well of a petri dish. The technique involves the sample to be analyzed on the support with an appropriate fixing material such as acetone and incubating the sample on the slide with a monoclonal antibody of the invention. After washing with an appropriate buffer such as, for example, phosphate buffered saline, the support is contacted with a labeled specific binding partner for the analyte in the sample. After incubation as desired, the slide is washed a second time with an aqueous buffer and the determination is made of the binding of the labeled monoclonal antibody to the analyte. If the label is fluorescent, the slide may be covered with a fluorescent antibody mounting fluid on a cover slip and then examined with a fluorescent microscope to determine the extent of binding. On the other hand, the label can be an enzyme conjugated to the monoclonal antibody of the invention and the the extent of binding can be determined by examining the slide for the presence of enzyme activity, which may be indicated by the formation of a precipitate, a color, or the like. A particular example of an assay utilizing the present antibodies is a double determinant ELISA assay. A support such as, e.g., a glass or vinyl plate, is coated with antibody specific for Carcinoma-related peptide by conventional techniques. The support is contacted with the sample suspected of containing Carcinoma-related peptides, usually in an aqueous medium. After an incubation period from 30 seconds to 12 hours, the support is separated from the medium, washed to remove unbound Carcinoma-related peptides with, for example, water or an aqueous buffered medium, and contacted with an antibody specific for Carcinoma-related peptides, again usually in an aqueous medium. The antibody is labeled with an enzyme directly or indirectly such as, e.g., horseradish peroxidase or alkaline phosphatase. After incubation, the support is separated from the medium, and washed as above. The enzyme activity of the support or the aqueous medium is determined. This enzyme activity is related to the amount of Carcinoma-related peptide in the sample.

The invention also includes kits, e.g., diagnostic assay kits, for carrying out the methods disclosed above. In one embodiment, the kit comprises in packaged combination (a) a monoclonal antibody more specifically defined above and (b) a conjugate of a specific binding partner for the above monoclonal antibody and a label capable of producing a detectable signal. The reagents may also include ancillary agents such as buffering agents and protein stabilizing agents, e.g., polysaccharides and the like. The kit may further include, where necessary, other members of the signal producing system of which system the label is a member, agents for reducing background interference in a test, control reagents, apparatus for conducting a test, and the like. In another embodiment, the diagnostic kit comprises a conjugate of monoclonal antibody of the invention and a label capable of producing a detectable signal. Ancillary agents as mentioned above may also be present.

In further aspects, the invention relates to the use of peptides of the invention in methods of treatment of signal transmission disorders, particularly neurological disorders. Peptides of the invention may display structural similarity to signalling peptides, particularly neurotransmitters. The peptides of SEQ ID NOS 11 to 18 may thus be useful in the modulation of signal transmission, and thus in the treatment of neurological disorders involving aberrant signal transmission. In one aspect, the invention also comprises method of drug screening comprising identifying test compounds that inhibit or stimulate the activity of a peptide of SEQ ID NOS 11 to 18. In one aspect, an antagonist of a peptide of the invention is used for the treatment of a central nervous system disorder, such as for example a mood disorder, a psychotic disorder, an anxiety disorder or a neurodegenerative disorder. In other aspects, an agonist or antagonist of the peptides of the invention is useful for the treatment of a gastrointestinal disorder, a respiratory disorder or a carcinoma. In other aspects, the invention comprises administering a peptide of the invention to a mammal suffering from a neurological disorder or preferably from a neurotransmitter deficiency.

Among neurotransmitters, the most common mechanism of cleaving a propeptide involves endoproteolytic processing at pairs of basic amino acids, either KR, RR, RK, or KK, in decreasing order of occurrence. The peptides disclosed in SEQ ID NOS 11 to 18 thus comprise mature peptide forms. However, some peptides undergo cleavage at a monobasic site, R and less common is cleavage at a monobasic K site or a nonbasic sites. Cleavage usually occurs on the C terminal side of the paired basic residues. The pancreatic proteolytic enzyme, trypsin, cleaves dibasic residues. The cleavage may be carried out by different enzymes in different tissues.

Neuropeptides of the invention may also possess C-terminal amidation. Many neuropeptdies have an amidated C terminal, which is derived from a glycine residue in the immature peptides. The conversion is a two step process: peptidyl glycine is converted to peptidyl alpha-hyroxyglycine by peptidyl glycine alpha-hydroxylating monooxygenase (PHM), then this is coverted to the peptidyl amide by peptidyl alpha-hydroxyglycing alha-amidatin lyase (PAL) with the simultaneous production of glyoxylic acid. The peptide of Seq ID NO 12 may be amidated at amino acid residues 14 to 17; the peptide of SEQ ID NO 13 may be amidated at amino acid residue 14.

In further embodiments, a peptide of the invention may undergo N-terminal cyclization: In this form of post translational processing, an N-terminal glutamine is coverted to a pyroglutamic acid residue. The enzyme that does this, glutaminyl cyclase, has not been fully characterized. However, during the cyclization there is an empirical loss of NH3, so there is probably production of ammonia, ammonium, or a further amidation involved as well.

### Recombinant Manufacture of Carcinoma-Related Peptides

The polynucleotide sequences described herein can be used in recombinant DNA molecules that direct the expression of the corresponding peptides in appropriate host cells. Because of the degeneracy in the genetic code, other DNA sequences may encode the equivalent amino acid sequence, and may be used to clone and express the Carcinoma-related peptides. Codons preferred by a particular host cell may be selected and substituted into the naturally occurring nucleotide sequences, to increase the rate and/or efficiency of expression. The nucleic acid (e.g., cDNA or genomic DNA) encoding the desired Carcinoma-related peptide may be inserted into a replicable vector for cloning (amplification of the DNA), or for expression. The peptide can be expressed recombinantly in any of a number of expression systems according to methods known in the art (Ausubel, et al., editors, Current Protocols in Molecular Biology, John Wiley & Sons, New York, 1990). Appropriate host cells include yeast, bacteria, archebacteria, fungi, and insect and animal cells, including mammalian cells, for example primary cells, including stem cells, including, but not limited to bone marrow stem cells. More specifically, these include, but are not limited to, microorganisms such as bacteria transformed with recombinant bacteriophage, plasmid or cosmid DNA expression vectors, and yeast transformed with yeast expression vectors. Also included, are insect cells infected with a recombinant insect virus (such as baculovirus), and mammalian expression systems. The nucleic acid sequence to be expressed may be inserted into the vector by a variety of procedures. In general, DNA is inserted into an appropriate restriction endonuclease site using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The Carcinoma-related peptides of the present invention are produced by culturing a host cell transformed with an expression vector containing a nucleic acid encoding a carcinoma-related peptide, under the appropriate conditions to induce or cause expression of the protein. The conditions appropriate for peptide expression will vary with the choice of the expression vector and the host cell, and will be easily ascertained by one skilled in the art through routine experimentation. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield.

A host cell strain may be chosen for its ability to modulate the expression of the inserted sequences or to process the expressed protein in the desired fashion. Such modifications of the protein include, but are not limited to, acetylation, carboxylation, glycosylation, phosphorylation, lipidation and acylation. Post-translational processing, which cleaves a “prepro” form of the protein, may also be important for correct insertion, folding and/or function. By way of example, host cells such as CHO, HeLa, BHK, MDCK, 293, W138, etc. have specific cellular machinery and characteristic mechanisms for such post-translational activities and may be chosen to ensure the correct modification and processing of the introduced, foreign protein. Of particular interest are Drosophila melangastev cells, Sacchavomyces cevevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines, Jukat cells, human cells and other primary cells.

The nucleic acid encoding a peptide must be “operably linked” by placing it into a functional relationship with another nucleic acid sequence. For example, DNA for a presequence or secretory leader is operably linked to DNA for a peptide if it is expressed as a preprotein that participates in the secretion of the peptide; a promoter or enhancer is operably linked to a coding sequence if it affects the transcription of the sequence; or a ribosome binding site is operably linked to a coding sequence if it is positioned so as to facilitate translation. Generally, “operably linked” DNA sequences are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. Promoter sequences encode either constitutive or inducible promoters. The promoters may be either naturally occurring promoters or hybrid promoters. Hybrid promoters, which combine elements of more than one promoter, are also known in the art, and are useful in the present invention. The expression vector may comprise additional elements, for example, the expression vector may have two replication systems, thus allowing it to be maintained in two organisms, for example in mammalian or insect cells for expression and in a procaryotic host for cloning and amplification. Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2: plasmid origin is suitable for yeast, and various viral origins (SV40, polyoma, adenovirus, VSV or BPV) are useful for cloning vectors in mammalian cells. Further, for integrating expression vectors, the expression vector contains at least one sequence homologous to the host cell genome, and preferably, two homologous sequences which flank the expression construct. The integrating vector may be directed to a specific locus in the host cell by selecting the appropriate homologous sequence for inclusion in the vector. Constructs for integrating vectors are well known in the art.

Preferably, the expression vector contains a selectable marker gene to allow the selection of transformed host cells. Selection genes are well known in the art and will vary with the host cell used. Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available for from complex media, e.g., the gene encoding D-alanine racemase for Bacilli.

Host cells transformed with a nucleotide sequence encoding a Carcinoma-related peptide may be cultured under conditions suitable for the expression and recovery of the encoded protein from cell culture. The protein produced by a recombinant cell may be secreted, membrane-bound, or contained intracellularly depending on the sequence and/or the vector used. As will be understood by those of skill in the art, expression vectors containing polynucleotides encoding the carcinoma-related peptide can be designed with signal sequences which direct secretion of the carcinoma-related peptide through a prokaryotic or eukaryotic cell membrane. The desired Carcinoma-related peptide may be produced recombinantly not only directly, but also as a fusion peptide with a heterologous peptide, which may be a signal sequence or other peptide having a specific cleavage site at the N-terminus of the mature protein or peptide. In general, the signal sequence may be a component of the vector, or it may be a part of the carcinoma-related peptide-encoding DNA that is inserted into the vector. The signal sequence may be a prokaryotic signal sequence selected, for example, from the group of the alkaline phosphatase, penicillinase, 1pp, or heat-stable enterotoxin II leaders. For yeast secretion the signal sequence may be, e.g., the yeast invertase leader, alpha factor leader (including Saccharomyces and Kluyveromyces a-factor leaders, the latter described in U.S. Pat. No. 5,010,182), or acid phosphatase leader, the C. albicans glucoamylase leader (EP 362,179 published Apr. 4, 1990), or the signal described in WO 90113646 published Nov. 15, 1990. In mammalian cell expression, mammalian signal sequences may be used to direct secretion of the protein, such as signal sequences from secreted peptides of the same or related species, as well as viral secretory leaders. According to the expression system selected, the coding sequence is inserted into an appropriate vector, which in turn may require the presence of certain characteristic “control elements” or “regulatory sequences.” Appropriate constructs are known generally in the art (Ausubel, et al., 1990) and, in many cases, are available from commercial suppliers such as Invitrogen (San Diego, Calif.), Stratagene (La Jolla, Calif.), Gibco BRL (Rockville, Md.) or Clontech (Palo Alto, Calif.).

Expression in Bacterial Systems.

Transformation of bacterial cells may be achieved using an inducible promoter such as the hybrid lacZ promoter of the “BLUESCRIPT” Phagemid (Stratagene) or “pSPORT1” (Gibco BRL). In addition, a number of expression vectors may be selected for use in bacterial cells to produce cleavable fusion proteins that can be easily detected and/or purified, including, but not limited to “BLUESCRIPT” (a-galactosidase; Stratagene) or pGEX (glutathione S-transferase; Promega, Madison, Wis.). A suitable bacterial promoter is any nucleic acid sequence capable of binding bacterial RNA polymerase and initiating the downstream (3′) transcription of the coding sequence of the peptide into mRNA. A bacterial promoter has a transcription initiation region which is usually placed proximal to the 5′ end of the coding sequence. This transcription initiation region typically includes an RNA polymerase binding site and a transcription initiation site. Sequences encoding metabolic pathway enzymes provide particularly useful promoter sequences. Examples include promoter sequences derived from sugar metabolizing enzymes, such as galactose, lactose and maltose, and sequences derived from biosynthetic enzymes such as tryptophan. Promoters from bacteriophage may also be used and are known in the art. In addition, synthetic promoters and hybrid promoters are also useful; for example, the tat promoter is a hybrid of the trp and lac promoter sequences. Furthermore, a bacterial promoter can include naturally occurring promoters of non-bacterial origin that have the ability to bind bacterial RNA polymerase and initiate transcription. An efficient ribosome binding site is also desirable. The expression vector may also include a signal peptide sequence that provides for secretion of the Carcinoma-related peptide in bacteria. The signal sequence typically encodes a signal peptide comprised of hydrophobic amino acids which direct the secretion of the protein from the cell, as is well known in the art. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include drug resistance genes such as ampicillin, chloramphenicol, erythromycin, kanamycin, neomycin and tetracycline. Selectable markers also include biosynthetic genes, such as those in the histidine, tryptophan and leucine biosynthetic pathways. When large quantities of Carcinoma-related peptides are needed, e.g., for the induction of antibodies, vectors which direct high level expression of fusion proteins that are readily purified may be desirable. Such vectors include, but are not limited to, multifunctional E. coli cloning and expression vectors such as BLUESCRIPT (Stratagene), in which the Carcinoma-related peptide coding sequence may be ligated into the vector in-frame with sequences for the amino-terminal Met and the subsequent 7 residues of beta-galactosidase so that a hybrid protein is produced; PIN vectors [Van Heeke & Schuster JBiol Chem 264:5503-5509 1989)]; PET vectors (Novagen, Madison Wis.); and the like. Expression vectors for bacteria include the various components set forth above, and are well known in the art. Examples include vectors for Bacillus subtilis, E. coli, Streptococcus cvemovis, and Streptococcus lividans, among others. Bacterial expression vectors are transformed into bacterial host cells using techniques well known in the art, such as calcium chloride mediated transfection, electroporation, and others.

Expression in Yeast.

Yeast expression systems are well known in the art, and include expression vectors for Sacchavomyces cevevisiae, Candida albicans and C. maltosa, Hansenula polymovpha, Kluyvevomyces fvagilis and K. lactis, Pichia guillevimondii and P pastoris, Schizosaccha-vomyces pombe, and Yavvowia lipolytica. Examples of suitable promoters for use in yeast hosts include the promoters for 3-phosphoglycerate kinase [Hitzeman et al., J. Biol. Chem. 255:2073 (1980)] or other glycolytic enzymes [Hess et al., J. Adv. Enzyme Reg. 7:149 (1968); Holland, Biochemistry 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, tri osephosphate isomerase, phosphoglucose isomerase, alpha factor, the ADH2IGAPDH promoter, glucokinase alcohol oxidase, and PGH. [See, for example, Ausubel, et al., 1990; Grant et al., Methods in Enzymology 153:516-544, (1987)]. Other yeast promoters, which are inducible have the additional advantage of transcription controlled by growth conditions, include the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast selectable markers include ADE2. HIS4. LEU2. TRP1. and ALG7, which confers resistance' to tunicamycin; the neomycin phosphotransferase gene, which confers resistance to G418; and the CUP1 gene, which allows yeast to grow in the presence of copper ions. Yeast expression vectors can be constructed for intracellular production or secretion of a peptide from the DNA encoding the carcinoma-related peptide of interest. For example, a selected signal peptide and the appropriate constitutive or inducible promoter may be inserted into suitable restriction sites in the selected plasmid for direct intracellular expression of the Carcinoma-related peptide. For secretion of the peptide, DNA encoding the Carcinoma-related peptide can be cloned into the selected plasmid, together with DNA encoding the promoter, the yeast alpha-factor secretory signal/leader sequence, and linker sequences (as needed), for expression of the Carcinoma-related peptide. Yeast cells, can then be transformed with the expression plasmids described above, and cultured in an appropriate fermentation media. The protein produced by such transformed yeast can then be concentrated by precipitation with 10% trichloroacetic acid and analyzed following separation by SDS-PAGE and staining of the gels with Coomassie Blue stain. The recombinant peptide can subsequently be isolated and purified from the fermentation medium by techniques known to those of skill in the art.

Expression in Mammalian Systems.

The Carcinoma-related peptides may be expressed in mammalian cells. Mammalian expression systems are known in the art, and include retroviral vector mediated expression systems. Mammalian host cells may be transformed with any of a number of different viral-based expression systems, such as adenovirus, where the coding region can be ligated into an adenovirus transcription/translation complex consisting of the late promoter and tripartite leader sequence. Insertion in a nonessential E1 or E3 region of the viral genome results in a viable virus capable of expression of the peptide of interest in infected host cells. A preferred expression vector system is a retroviral vector system such as is generally described in PCT/US97/01019 and PCT/US97/101048. Suitable mammalian expression vectors contain a mammalian promoter which is any DNA sequence capable of binding mammalian RNA polymerase and initiating the downstream (3′) transcription of a coding sequence for carcinoma-related peptide into mRNA. A promoter will have a transcription initiating region, which is usually placed proximal to the 5′ end of the coding sequence, and a TATA box, using a located 25-30 base pairs upstream of the transcription initiation site. The TATA box is thought to direct RNA polymerase II to begin RNA synthesis at the correct site. A mammalian promoter will also contain an upstream promoter element (enhancer element), typically located within 100 to 200 base pairs upstream of the TATA box. An upstream promoter element determines the rate at which transcription is initiated and can act in either orientation. Of particular use as mammalian promoters are the promoters from mammalian viral genes, since the viral genes are often highly expressed and have a broad host range. Examples include promoters obtained from the genomes of viruses such as polyoma virus, fowlpox virus (UK 2,211,504 published Jul. 5, 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems. Transcription of a DNA encoding a Carcinoma-related peptide by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, a-fetoprotein, and insulin). Typically, however, one will use an enhancer from a eukaryotic cell virus. Examples include the SV40 enhancer, the cytomegalovirus early promoter enhancer, the polyoma enhancer on the late side of the replication origin, and adenovirus enhancers. The enhancer is preferably located at a site 5′ from the promoter. In general, the transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Long term, high-yield production of recombinant proteins can be effected in a stable expression system. Expression vectors which contain viral origins of replication or endogenous expression elements and a selectable marker gene may be used for this purpose. Appropriate vectors containing selectable markers for use in mammalian cells are readily available commercially and are known to persons skilled in the art. Examples of such selectable markers include, but are not limited to herpes simplex virus thymi-dine kinase and adenine phosphoribosyltransferase for use in tk- or hprt-cells, respectively. The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

Expression in Insect Cells.

Carcinoma-related peptides may also be produced in insect cells. Expression vectors for the transformation of insect cells, and in particular, baculovirus-based expression vectors, are well known in the art. In one such system, the peptide-encoding DNA is fused upstream of an epitope tag contained within a baculovirus expression vector. Autographa califovnica nuclear polyhedrosis virus (AcNPV) is used as a vector to express foreign genes in Spodoptevu fmgipevdu Sf9 cells or in Trichoplusia larvae. The peptide-encoding sequence is cloned into a nonessential region of the virus, such as the polyhedrin gene, and placed under control of the polyhedrin promoter. Successful insertion of a peptide-encoding sequence will render the polyhedrin gene inactive and produce recombinant virus lacking coat protein coat. The recombinant viruses are then used to infect S. fmgipevdu cells or Trichoplusia larvae in which the carcinoma-related peptide is expressed [Smith et al., J. Wol. 46:584 (1994); Engelhard E K et al., Pvoc. Nat. Acad. Sci. 91:3224-3227 (1994)]. Suitable epitope tags for fusion to the carcinoma-related peptide-encoding DNA include poly-his tags and immunoglobulin tags (like Fc regions of IgG). A variety of plasmids may be employed, including commercially available plasmids such as pVL1393 (Novagen). Briefly, the carcinoma-related peptide-encoding DNA or the desired portion of the carcinoma-related peptide-encoding DNA is amplified by PCR with primers complementary to the 5′ and 3′ regions. The 5′ primer may incorporate flanking restriction sites. The PCR product is then digested with the selected restriction enzymes and subcloned into an expression vector. Recombinant baculovirus is generated by co-transfecting the above plasmid and BaculoGold™ virus DNA (Pharmingen) into Spodopteva fvugipevda (“Sf9”) cells (ATCC CRL 1711) using lipofectin (commercially available from GIBCO-BRL), or other methods known to those of skill in the art. Virus is produced by day 4-5 of culture in Sf9 cells at 28° C., and used for further amplifications. Procedures are performed as further described in O'Reilley et al., BACULOVIRUS EXPRESSION VECTORS: A LABORATORY MANUAL, Oxford University Press (1994). Extracts may be prepared from recombinant virus-infected Sf9 cells as described in Rupert et al., Nature 362:175-179 (1993). Alternatively, expressed epitope-tagged Carcinoma-related peptides can be purified by affinity chromatography, or for example, purification of an IgG tagged (or Fc tagged) Carcinoma-related peptide can be performed using chromatography techniques, including Protein A or protein G column chromatography.

Evaluation of Gene Expression.

Gene expression may be evaluated in a sample directly, for example, by standard techniques known to those of skill in the art, e.g., Southern blotting for DNA detection, Northern blotting to determine the transcription of mRNA, dot blotting (DNA or RNA), or in situ hybridization, using an appropriately labeled probe, based on the sequences provided herein. Alternatively, antibodies may be used in assays for detection of nucleic acids, such as specific duplexes, including DNA duplexes, RNA duplexes, and DNA-RNA hybrid duplexes or DNA-protein duplexes. Such antibodies may be labeled and the assay carried out where the duplex is bound to a surface, so that upon the formation of duplex on the surface, the presence of antibody bound to the duplex can be detected. Gene expression, alternatively, may be measured by immunohistochemical staining of cells or tissue sections and assay of cell culture or body fluids, to directly evaluate the expression of carcinoma-related peptides. Antibodies useful for such immunological assays may be either monoclonal or polyclonal, and may be prepared against a native sequence carcinoma-related peptide based on the DNA sequences provided herein.

Purification of Expressed Protein.

Expressed Carcinoma-related peptides may be purified or isolated after expression, using any of a variety of methods known to those skilled in the art. The appropriate technique will vary depending upon what other components are present in the sample. Contaminant components that are removed by isolation or purification are materials that would typically interfere with diagnostic or therapeutic uses for the peptide, and may include enzymes, hormones, and other solutes. The purification step(s) selected will depend, for example, on the nature of the production process used and the particular Carcinoma-related peptide produced. An Carcinoma-related peptide or protein may be recovered from culture medium or from host cell lysates. If membrane-bound, it can be released from the membrane using a suitable detergent solution (e.g. Triton-X 100) or by enzymatic cleavage. Alternatively, cells employed in expression of Carcinoma-related peptides can be disrupted by various physical or chemical means, such as freeze-thaw cycling, sonication, mechanical disruption, or by use of cell lysing agents. Exemplary purification methods include, but are not limited to, ion-exchange column chromatography; chromatography using silica gel or a cation-exchange resin such as DEAE; gel filtration using, for example, Sephadex G-75; protein A Sepharose columns to remove contaminants such as IgG; chromatography using metal chelating columns to bind epitope-tagged forms of the Carcinoma-related peptide; ethanol precipitation; reverse phase HPLC; chromatofocusing; SDS-PAGE; and ammonium sulfate precipitation. Ordinarily, an isolated Carcinoma-related peptide will be prepared by at least one purification step. For example, the carcinoma-related peptide may be purified using a standard anti-carcinoma-related peptide antibody column. Ultrafiltration and dialysis techniques, in conjunction with protein concentration, are also useful (see, for example, Scopes, R., PROTEIN PURIFICATION, Springer-Verlag, New York, N.Y., 1982). The degree of purification necessary will vary depending on the use of the carcinoma-related peptide. In some instances no purification will be necessary. Once expressed and purified as needed, the Carcinoma-related peptides and nucleic acids of the present invention are useful in a number of applications, as detailed below.

Labeling of Expressed Protein.

The nucleic acids, proteins and antibodies of the invention may be labeled. By labeled herein is meant that a compound has at least one element, isotope or chemical compound attached to enable the detection of the compound. In general, labels fall into three classes: a) isotopic labels, which may be radioactive or heavy isotopes; b) immune labels, which may be antibodies or antigens; and c) colored or fluorescent dyes. The labels may be incorporated into the compound at any position that does not interfere with the biological activity or characteristic of the compound which is being detected.

### Chemical Manufacture of Carcinoma-Related Peptide Compositions

peptides of the invention are synthesized by standard techniques, e.g. Stewart and Young, Solid Phase peptide Synthesis, 2nd Ed. (Pierce Chemical Company, Rockford, Ill., 1984). Preferably, a commercial peptide synthesizer is used, e.g. Applied Biosystems, Inc. Foster City, Calif.) model 430A, and peptides of the invention may be assembled from multiple, separately synthesized and purified peptides in a convergent synthesis approach, e.g. Kent et al, U.S. Pat. No. 6,184,344 and Dawson and Kent, Annu. Rev. Biochem., 69: 923-960 (2000). The peptides of the invention may be assembled by solid phase synthesis on a cross-linked polystyrene support starting from the carboxyl terminal residue and adding amino acids in a stepwise fashion until the entire peptide has been formed. The following references are guides to the chemistry employed during synthesis: Schnolzer et al, Int. J. peptide Protein Res., 40: 180-193 (1992); Merrifield, J. Amer. Chem. Soc., Vol. 85, pg. 2149 (1963); Kent et al., pg 185, in peptides 1984, Ragnarsson, Ed; (Almquist and Weksell, Stockholm, 1984); Kent et al., pg. 217 in peptide Chemistry 84, Izumiya, Ed. (Protein Research Foundation, B. H. Osaka, 1985); Merrifield, Science, Vol. 232, pgs. 341-347 (1986); Kent, Ann. Rev. Biochem., Vol. 57, pgs. 957-989 (1988), and references cited in these latter two references.

Preferably, chemical synthesis of peptides of the invention is carried out by the assembly of oligo peptides by native chemical ligation, as described by Dawson et al, Science, 266: 776-779 (1994) and Kent el al, U.S. Pat. No. 6,184,344. Briefly, in the approach a first oligo peptide is provided with an N-terminal cysteine having an unoxidized sulfhydryl side chain, and a second oligo peptide is provided with a C-terminal thioester. The unoxidized sulfhydryl side chain of the N-terminal cysteine is then condensed with the C-terminal thioester to produce an intermediate oligo peptide which links the first and second oligo peptides with a β-aminothioester bond. The β-aminothioester bond of the intermediate oligo peptide then undergoes an intramolecular rearrangement to produce the oligo peptide product which links the first and second oligo peptides with an amide bond. Preferably, the N-terminal cysteine of internal fragments are protected from undesired cyclization and/ro concatenation reactions by a cyclic thiazolidine protecting group as described below. Preferably, such cyclic thiazolidine protecting group is a thioprolinyl group.

Oligo peptides having a C-terminal thioester may be produced as described in the following references, which are incorporated by reference: Kent et al, U.S. Pat. No. 6,184,344; Tam et al, Proc. Natl. Acad. Sci., 92: 12485-12489 (1995); Blake, Int. J. peptide Protein Res., 17: 273 (1981); Canne et al, Tetrahedron Letters, 36: 1217-1220 (1995); Hackeng et al, Proc. Natl. Acad. Sci., 94: 7845-7850 (1997); or Hackeng et al, Proc. Natl. Acad. Sci., 96: 10068-10073 (1999). Preferably, the method described by Hackeng et al (1999) is employed. Briefly, oligo peptides are synthesized on a solid phase support (described below) typically on a 0.25 mmol scale by using the in situ neutralization/HBTU activation procedure for Boc chemistry dislosed by Schnolzer et al, Int. J. peptide Protein Res., 40: 180-193 (1992), which reference is incorporated herein by reference. (HBTU is 2-(1H-benzotriazol-1-yl)-1,1,3,3-tetramethyluronium hexafluorophosphate and Boc is tert-butoxycarbonyl). Each synthetic cycle consists of N^{α}-Boc removal by a 1- to 2-minute treatment with neat TFA, a 1-minute DMF flow wash, a 10- to 20-minute coupling time with 1.0 mmol of preactivated Boc-amino acid in the presence of DIEA, and a second DMF flow wash: (TFA is trifluoroacetic acid, DMF is N,N-dimethylformamide, and DIEA is N,N-diisopropylethylamine). N^{α}-Boc-amino acids (1.1 mmol) are preactivated for 3 minutes with 1.0 mmol of HBTU (0.5 M in DMF) in the presence of excess DIEA (3 mmol). After each coupling step, yields are determined by measuring residual free amine with a conventional quantitative ninhydrin assay, e.g. as disclosed in Sarin et al, Anal. Biochem., 117: 147-157 (1981). After coupling of Gln residues, a DCM flow wash is used before and after deprotection by using TFA, to prevent possible high-temperature (TFA/DMF)-catalyzed pyrrolidone formation. After chain assembly is completed, the oligo peptides are deprotected and cleaved from the resin by treatment with anhydrous HF for 1 hour at 0° C. with 4% p-cresol as a scavenger. The imidazole side-chain 2,4-dinitrophenyl (dnp) protecting groups remain on the His residues because the dnp-removal procedure is incompatible with C-terminal thioester groups. However, dnp is gradually removed by thiols during the ligation reaction. After cleavage, oligo peptides are precipitated with ice-cold diethylether, dissolved in aqueous acetonitrile, and lyophilized.

Thioester oligo peptides described above are preferably synthesized on a trityl-associated mercaptopropionic acid-leucine (TAMPAL) resin, made as disclosed by Hackeng et al (1999), or comparable protocol. Briefly, N^{α}-Boc-Leu (4 mmol) is activated with 3.6 mmol of HBTU in the presence of 6 mmol of DIEA and coupled for 16 minutes to 2 mmol of p-methylbenzhydrylamine (MBHA) resin, or the equivalent. Next, 3 mmol of S-trityl mercaptopropionic acid is activated with 2.7 mmol of HBTU in the presence of 6 mmol of DIEA and coupled for 16 minutes to Leu-MBHA resin. The resulting TAMPAL resin can be used as a starting resin for peptide-chain assembly after removal of the trityl protecting group with two 1-minute treatments with 3.5% triisopropylsilane and 2.5% H₂O in TFA. The thioester bond can be formed with any desired amino acid by using standard in situ-neutralization peptide coupling protocols for 1 hour, as disclosed in Schnolzer et al (cited above). Treatment of the final oligo peptide with anhydrous HF yields the C-terminal activated mercaptopropionic acid-leucine (MPAL) thioester oligo peptides.

Preferably, thiazolidine-protected thioester oligo peptide intermediates are used in native chemical ligation under conditions as described by Hackeng et al (1999), or like conditions. Briefly, 0.1 M phosphate buffer (pH 8.5) containing 6 M guanidine, 4% (vol/vol) benzylmercaptan, and 4% (vol/vol) thiophenol is added to dry peptides to be ligated, to give a final peptide concentration of 1-3 mM at about pH 7, lowered because of the addition of thiols and TFA from the lyophilized peptide. Preferably, the ligation reaction is performed in a heating block at 37° C. and is periodically vortexed to equilibrate the thiol additives. The reaction may be monitored for degree of completion by MALDI-MS or HPLC and electrospray ionization MS.

After a native chemical ligation reaction is completed or stopped, the N-terminal thiazolidine ring of the product is opened by treatment with a cysteine deprotecting agent, such as O-methylhydroxylamine (0.5 M) at pH 3.5-4.5 for 2 hours at 37° C., after which a 10-fold excess of Tris-(2-carboxyethyl)-phosphine is added to the reaction mixture to completely reduce any oxidizing reaction constituents prior to purification of the product by conventional preparative HPLC. Preferably, fractions containing the ligation product are identified by electrospray MS, are pooled, and lyophilized.

After the synthesis is completed and the final product purified, the final peptide product may be refolded by conventional techniques, e.g. Creighton, Meth. Enzymol., 107: 305-329 (1984); White, Meth. Enzymol., 11: 481-484 (1967); Wetlaufer, Meth. Enzymol., 107: 301-304 (1984); and the like. Preferably, a final product is refolded by air oxidation by the following, or like: The reduced lyophilized product is dissolved (at about 0.1 mg/mL) in 1 M guanidine hydrochloride (or like chaotropic agent) with 100 mM Tris, 10 mM methionine, at pH 8.6. After gentle overnight stirring, the re-folded product is isolated by reverse phase HPLC with conventional protocols.

### Anti-Carcinoma-Related Peptide Antibodies

The present invention further provides anti-carcinoma-related peptide antibodies. The antibodies of the present invention include polyclonal, monoclonal, humanized, bispecific, and heteroconjugate antibodies.

Polyclonal Antibodies.

The anti-carcinoma-related peptide antibodies of the present invention may be polyclonal antibodies. Methods of preparing polyclonal antibodies are known to the skilled artisan. Such polyclonal antibodies can be produced in a mammal, for example, following one or more injections of an immunizing agent, and preferably, an adjuvant. Typically, the immunizing agent and/or adjuvant will be injected into the mammal by a series of subcutaneous or intraperitoneal injections. The immunizing agent may include a peptide or a fusion protein thereof. It may be useful to conjugate the antigen to a protein known to be immunogenic in the mammal being immunized. Examples of such immunogenic proteins include, but are not limited to, keyhole limpet hemocyanin (KLH), serum albumin, bovine thyroglobulin, and soybean trypsin inhibitor. Adjuvants include, for example, Freund's complete adjuvant and MPL-TDM adjuvant (monophosphoryl Lipid A, synthetic trehalose dicoryno-mycolate). The immunization protocol may be determined by one skilled in the art based on standard protocols or by routine experimentation.

Monoclonal Antibodies.

Alternatively, the anti-carcinoma-related peptide antibodies may be monoclonal antibodies. Monoclonal antibodies may be produced by hybridomas, wherein a mouse, hamster, or other appropriate host animal, is immunized with an immunizing agent to elicit lympho-cytes that produce or are capable of producing antibodies that will specifically bind to the immunizing agent [Kohler and Milstein, Nature 256:495 (1975)]. Alternatively, the lymphocytes may be immunized in vitro. The immunizing agent will typically include the peptide or a fusion protein thereof. Generally, spleen cells or lymph node cells are used if non-human mammalian sources are desired, or peripheral blood lymphocytes (“PBLs”) are used if cells of human origin. The lymphocytes are fused with an immortalized cell line using a suitable fusing agent, such as polyethylene glycol, to produce a hybridoma cell [Goding, MONOCLONAL ANTIBODIES: PRINCIPLES AND PRACTICE, Academic Press, pp. 59-103 (1986)]. In general, immortalized cell lines are transformed mammalian cells, for example, myeloma cells of rat, mouse, bovine or human origin. The hybridoma cells are cultured in a suitable culture medium that preferably contains one or more substances that inhibit the growth or survival of unfused, immortalized cells. For example, if the parental cells lack the enzyme hypoxanthine guanine phosphoribosyl transferase (HGPRT), the culture medium for the hybridomas typically will include hypoxanthine, aminopterin, and thymidine (HAT), substances which prevent the growth of HGPRT-deficient cells. Preferred immortalized cell lines are those that fuse efficiently, support stable high level production of antibody, and are sensitive to a medium such as HAT medium. More preferred immortalized cell lines are murine or human myeloma lines, which can be obtained, for example, from the American Type Culture Collection (ATCC), Rockville, Md. Human myeloma and mouse-human heteromyeloma cell lines also have been described for the production of human monoclonal antibodies [Kozbor, J. Zmmunol. 133:3001 (1984); Brodeur et al., Monoclonal Antibody Production Techniques and Applications, Marcel Dekker, Inc., New York, pp. 51-63 (1987)].

The culture medium (supernatant) in which the hybridoma cells are cultured can be assayed for the presence of monoclonal antibodies directed against an peptide. Preferably, the binding specificity of monoclonal antibodies present in the hybridoma supernatant is determined by immunoprecipitation or by an in vitro binding assay, such as radioimmunoassay (RIA) or enzyme-linked immunoabsorbent assay (ELISA). Appropriate techniques and assays are known in the art. The binding affinity of the monoclonal antibody can, for example, be determined by the Scatchard analysis of Munson and Pollard, Anal. Biochem. 107:220 (1980). After the desired antibody-producing hybridoma cells are identified, the cells may be cloned by limiting dilution procedures and grown by standard methods [Goding, 1986]. Suitable culture media for this purpose include, for example, Dulbecco's Modified Eagle's Medium and RPMI-1640 medium. Alternatively, the hybridoma cells may be grown in vivo as ascites in a mammal. The monoclonal antibodies secreted by selected clones may be isolated or purified from the culture medium or ascites fluid by immunoglobulin purification procedures routinely used by those of skill in the art such as, for example, protein A-Sepharose, hydroxyl-apatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be isolated from the peptide-specific hybridoma cells and sequenced, e.g., by using oligonucleotide probes that are capable of binding specifically to genes encoding the heavy and light chains of murine antibodies. Once isolated, the DNA may be inserted into an expression vector, which is then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for the human heavy and light chain constant domains for the homologous murine sequences [Morrison et al., Proc. Nat. Acad. Sci. 81:6851-6855 (1984); Neuberger et al., Nature 312:604-608 (1984); Takeda et al., Nature 314:452-454 (1985)], or by covalently joining to the immunoglobulin coding sequence all or part of the coding sequence for a non-immunoglobulin peptide. The non-immunoglobulin peptide can be substituted for the constant domains of an antibody of the invention, or can be substituted for the variable domains of one antigen-combining site of an antibody of the invention to create a chimeric bivalent antibody. The antibodies may also be monovalent antibodies. Methods for preparing monovalent antibodies are well known in the art. For example, in vitro methods are suitable for preparing monovalent antibodies. Digestion of antibodies to produce fragments thereof, particularly, Fab fragments, can be accomplished using routine techniques known in the art.

Antibodies and antibody fragments characteristic of hybridomas of the invention can also be produced by recombinant means by extracting messenger RNA, constructing a cDNA library, and selecting clones which encode segments of the antibody molecule, e.g. Wall et al., Nucleic Acids Research, Vol. 5, pgs. 3113-3128 (1978); Zakut et al., Nucleic Acids Research, Vol. 8, pgs. 3591-3601 (1980); Cabilly et al., Proc. Natl. Acad. Sci., Vol. 81, pgs. 3273-3277 (1984); Boss et al., Nucleic Acids Research, Vol. 12, pgs. 3791-3806 (1984); Amster et al., Nucleic Acids Research, Vol. 8, pgs. 2055-2065 (1980); Moore et al., U.S. Pat. No. 4,642,334; Skerra et al, Science, Vol. 240, pgs. 1038-1041 (1988); and Huse et al, Science, Vol. 246, pgs. 1275-1281 (1989). In particular, such techniques can be used to produce interspecific monoclonal antibodies, wherein the binding region of one species is combined with non-binding region of the antibody of another species to reduce immunogenicity, e.g. Liu et al., Proc. Natl. Acad. Sci., Vol. 84, pgs. 3439-3443 (1987).

Both polyclonal and monoclonal antibodies can be screened by ELISA. As in other solid phase immunoassays, the test is based on the tendency of macromolecules to adsorb nonspecifically to plastic. The irreversibility of this reaction, without loss of immunological activity, allows the formation of antigen-antibody complexes with a simple separation of such complexes from unbound material. To titrate anti peptide serum, peptide conjugated to a carrier different from that used in immunization is adsorbed to the wells of a 96-well microtiter plate. The adsorbed antigen is then allowed to react in the wells with dilutions of anti-peptide serum. Unbound antibody is washed away, and the remaining antigen-antibody complexes are allowed to react with antibody specific for the IgG of the immunized animal. this second antibody is conjugated to an enzyme such as alkaline phosphatase. A visible colored reaction product produced when the enzyme substrate is added indicates which wells have bound anti peptide antibodies. The use of spectrophotometer readings allows better quantification of the amount of peptide-specific antibody bound. High-titer antisera yield a linear titration curve between 10⁻³ and 10⁻⁵ dilutions.

Carcinoma-Related Peptide Antibodies.

The invention includes peptides derived from peptide, and immunogens comprising conjugates between carriers and peptides of the invention. The term immnunogen as used herein refers to a substance which is capable of causing an immune response. The term carrier as used herein refers to any substance which when chemically conjugated to a peptide of the invention permits a host organism immunized with the resulting conjugate to generate antibodies specific for the conjugated peptide. Carriers include red blood cells, bacteriophages, proteins, or synthetic particles such as agarose beads. Preferably, carriers are proteins, such as serum albumin, gamma-globulin, keyhole limpet hemocyanin, thyroglobulin, ovalbumin, fibrinogen, or the like.

The general technique of linking synthetic peptides to a carrier is described in several references, e.g. Walter and Doolittle, “Antibodies Against Synthetic peptides,” in Setlow et al., eds., Genetic Engineering, Vol. 5, pgs. 61-91 (Plenum Press, New York, 1983); Green et al. Cell, Vol. 28, pgs. 477-487 (1982); Lerner et al., Proc. Natl. Acad. Sci., Vol. 78, pgs. 3403-3407 (1981); Shimizu et al., U.S. Pat. No. 4,474,754; and Ganfield et al., U.S. Pat. No. 4,311,639. Accordingly, these references are incorporated by reference. Also, techniques employed to link haptens to carriers are essentially the same as the above-referenced techniques, e.g. chapter 20 in Tijsseu Practice and Theory of Enzyme Immunoassays (Elsevier, New York, 1985). The four most commonly used schemes for attaching a peptide to a carrier are (1) glutaraldehyde for amino coupling, e.g. as disclosed by Kagan and Glick, in Jaffe and Behrman, eds. Methods of Hormone Radioimmunoassay, pgs. 328-329 (Academic Press, New York, 1979), and Walter et al. Proc. Natl. Acad. Sci., Vol. 77, pgs. 5197-5200 (1980); (2) water-soluble carbodiimides for carboxyl to amino coupling, e.g. as disclosed by Hoare et al., J. Biol. Chem., Vol. 242, pgs. 2447-2453 (1967); (3) bis-diazobenzidine (DBD) for tyrosine to tyrosine sidechain coupling, e.g. as disclosed by Bassiri et al., pgs. 46-47, in Jaffe and Behrman, eds. (cited above), and Walter et al. (cited above); and (4) maleimidobenzoyl-N-hydroxysuccinimide ester (MBS) for coupling cysteine (or other sulfhydryls) to amino groups, e.g. as disclosed by Kitagawa et al., J. Biochem. (Tokyo), Vol. 79, pgs. 233-239 (1976), and Lerner et al. (cited above). A general rule for selecting an appropriate method for coupling a given peptide to a protein carrier can be stated as follows: the group involved in attachment should occur only once in the sequence, preferably at the appropriate end of the segment. For example, BDB should not be used if a tyrosine residue occurs in the main part of a sequence chosen for its potentially antigenic character. Similarly, centrally located lysines rule out the glutaraldehyde method, and the occurrences of aspartic and glutamic acids frequently exclude the carbodiimide approach. On the other hand, suitable residues can be positioned at either end of chosen sequence segment as attachment sites, whether or not they occur in the “native” protein sequence. Internal segments, unlike the amino and carboxy termini, will differ significantly at the “unattached end” from the same sequence as it is found in the native protein where the peptide backbone is continuous. The problem can be remedied, to a degree, by acetylating the □-amino group and then attaching the peptide by way of its carboxy terminus. The coupling efficiency to the carrier protein is conveniently measured by using a radioactively labeled peptide, prepared either by using a radioactive amino acid for one step of the synthesis or by labeling the completed peptide by the iodination of a tyrosine residue. The presence of tyrosine in the peptide also allows one to set up a sensitive radioimmune assay, if desirable. Therefore, tyrosine can be introduced as a terminal residue if it is not part of the peptide sequence defined by the native peptide.

Preferred carriers are proteins, and preferred protein carriers include bovine serum albumin, myoglobulin, ovalbumin (OVA), keyhole limpet hemocyanin (KLH), or the like. peptides can be linked to KLH through cysteines by MBS as disclosed by Liu et al., Biochemistry, Vol. 18, pgs. 690-697 (1979). The peptides are dissolved in phosphate-buffered saline (pH 7.5), 0.1 M sodium borate buffer (pH 9.0) or 1.0 M sodium acetate buffer (pH 4.0). The pH for the dissolution of the peptide is chosen to optimize peptide solubility. The content of free cysteine for soluble peptides is determined by Ellman's method, Ellman, Arch. Biochem. Biophys., Vol. 82, pg. 7077 (1959). For each peptide, 4 mg KLH in 0.25 ml of 10 mM sodium phosphate buffer (pH 7.2) is reacted with 0.7 mg MBS (dissolved in dimethyl formamide) and stirred for 30 min at room temperature. The MBS is added dropwise to ensure that the local concentration of formamide is not too high, as KLH is insoluble in >30% formamide. The reaction product, KLH-MBS, is then passed through Sephadex G-25 equilibrated with 50 mM sodium phosphate buffer (pH 6.0) to remove free MBS, KLH recovery from peak fractions of the column eluate (monitored by OD280) is estimated to be approximately 80%. KLH-MBS is then reacted with 5 mg peptide dissolved 25 in 1 ml of the chosen buffer. The pH is adjusted to 7-7.5 and the reaction is stirred for 3 hr at room temperature. Coupling efficiency is monitored with radioactive peptide by dialysis of a sample of the conjugate against phosphate-buffered saline, and ranged from 8% to 60%. Once the peptide-carrier conjugate is available polyclonal or monoclonal antibodies are produced by standard techniques, e.g. as disclosed by Campbell, Monoclonal Antibody Technology (Elsevier, New York, 1984); Hurrell, ed. Monoclonal Hybridoma Antibodies: Techniques and Applications (CRC Press, Boca Raton, Fla., 1982); Schreier et al. Hybridoma Techniques (Cold Spring Harbor Laboratory, New York, 1980); U.S. Pat. No. 4,562,003; or the like. In particular, U.S. Pat. No. 4,562,003 is incorporated by reference.

Humanized Antibodies.

The anti-carcinoma-related peptide antibodies of the invention may further comprise humanized antibodies or human antibodies. The term “humanized antibody” refers to humanized forms of non-human (e.g., murine) antibodies that are chimeric antibodies, immunoglobulin chains or fragments thereof (such as Fv, Fab, Fab′, F(ab′), or other antigen-binding partial sequences of antibodies) which contain some portion of the sequence derived from non-human antibody. Humanized antibodies include human immunoglobulins in which residues from a complementary determining region (CDR) of the human immunoglobulin are replaced by residues from a CDR of a non-human species such as mouse, rat or rabbit having the desired binding specificity, affinity and capacity. In general, the humanized antibody will comprise substantially all of at least one, and generally two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature 321:522-525 (1986) and Presta, Cuvv. Op. Stvuct. Biol. 2:593-596 (1992)]. Methods for humanizing non-human antibodies are well known in the art. Generally, a humanized antibody has one or more amino acids introduced into it from a source which is non-human in order to more closely resemble a human antibody, while still retaining the original binding activity of the antibody. Methods for humanization of antibodies are further detailed in Jones et al., Nature 321:522-525 (1986); Riechmann et al., Nature 332:323-327 (1988); and Verhoeyen et al., Science 239:1534-1536 (1988). Such “humanized” antibodies are chimeric antibodies in that substantially less than an intact human variable domain has been substituted by the corresponding sequence from a non-human species.

Heteroconjugate Antibodies.

Heteroconjugate antibodies which comprise two covalently joined antibodies, are also within the scope of the present invention. Heteroconjugate antibodies may be prepared in vitro using known methods in synthetic protein chemistry, including those involving crosslinking agents. For example, immunotoxins may be prepared using a disulfide exchange reaction or by forming a thioether bond.

Bispecific Antibodies.

Bispecific antibodies have binding specificities for at least two different antigens. Such antibodies are monoclonal, and preferably human or humanized. One of the binding specificities of a bispecific antibody of the present invention is for a peptide, and the other one is preferably for a cell-surface protein or receptor or receptor subunit. Methods for making bispecific antibodies are known in the art, and in general, the recombinant production of bispecific antibodies is based on the co-expression of two immunoglobulin heavy-chain/light-chain pairs in hybridoma cells, where the two heavy chains have different specificities [Milstein and Cuello, Nature 305:537-539 (1983)]. Given that the random assortment of immunoglobulin heavy and light chains results in production of potentially ten different antibody molecules by the hybridomas, purification of the correct molecule usually requires some sort of affinity purification, e.g. affinity chromatography.

Antibody Antagonists.

Preferably, antagonists of the invention are derived from antibodies specific for a carcinoma-related peptide. More preferably, the antagonists of the invention comprise fragments or binding compositions specific for a carcinoma-related peptide. Antibodies comprise an assembly of peptide chains linked together by disulfide bridges. Two major peptide chains, referred to as the light chain and the heavy chain, make up all major structural classes (isotypes) of antibody. Both heavy chains and light chains are further divided into subregions referred to as—variable regions and constant regions. Heavy chains comprise a single variable region and three different constant regions, and light chains comprise a single variable region (different from that of the heavy chain) and a single constant region (different from those of the heavy chain). The variable regions of the heavy chain and light chain are responsible for the antibody's binding specificity. As used herein, the term “heavy chain variable region” means a poly peptide (1) which is from 110 to 125 amino acids in length, and (2) whose amino acid sequence corresponds to that of a heavy chain of a monoclonal antibody of the invention, starting from the heavy chain's N-terminal amino acid. Likewise, the term “light chain variable region” means a poly peptide (1) which is from 95 to 115 amino acids in length, and (2) whose amino acid sequence corresponds to that of a light chain of a monoclonal antibody of the invention, starting from the light chain's N-terminal amino acid. As used herein the term “monoclonal antibody” refers to homogeneous populations of immunoglobulins which are capable of specifically binding to a carcinoma-related peptide. As used herein the term “binding composition” means a composition comprising two polypeptide chains (1) which, when operationally associated, assume a conformation having high binding affinity for a carcinoma-related peptide peptide, and (2) which are derived from a hybridoma producing monoclonal antibodies specific for a carcinoma-related peptide peptide. The term “operationally associated” is meant to indicate that the two peptide chains can be positioned relative to one another for binding by a variety of means, including by association in a native antibody fragment, such as Fab or Fv, or by way of genetically engineered cysteine-containing peptide linkers at the carboxyl termini. Normally, the two polypeptide chains correspond to the light chain variable region and heavy chain variable region of a monoclonal antibody specific for a carcinoma-related peptide peptide. Preferably, antagonists of the invention are derived from monoclonal antibodies specific for a carcinoma-related peptide. Monoclonal antibodies capable of blocking, or neutralizing, a carcinoma-related peptide peptide are selected by their ability to inhibit carcinoma-related peptide-induced effects.

The use and generation of fragments of antibodies is also well known, e.g. Fab fragments: Tijssen, Practice and Theory of Enzyme Immunoassays (Elsevier, Amsterdam, 1985); and Fv fragments: Hochman et al. Biochemistry, Vol. 12, pgs. 1130-1135 (1973), Sharon et al., Biochemistry, Vol. 15, pgs. 1591-1594 (1976) and Ehrlich et al., U.S. Pat. No. 4,355,023; and antibody half molecules: Auditore-Hargreaves, U.S. Pat. No. 4,470,925.

### Uses of Polynucleotides Encoding Carcinoma-Related Peptides

Polynucleotide sequences (or the complements thereof) which encode Carcinoma-related peptides have various applications, including uses as hybridization probes, in chromosome and gene mapping, and in the generation of antisense RNA and DNA. In addition, peptide-encoding nucleic acids are useful as targets for pharmaceutical intervention, e.g. for the development of DNA vaccines, and for the preparation of Carcinoma-related peptides by recombinant techniques, as described herein. The polynucleotides described herein, including sequence variants thereof, can be used in diagnostic assays. Accordingly, diagnostic methods based on detecting the presence of such polynucleotides in body fluids or tissue samples are a feature of the present invention. Examples of nucleic acid based diagnostic assays in accordance with the present invention include, but are not limited to, hybridization assays, e.g., in situ hybridization, and PCR-based assays. Polynucleotides, including extended length polynucleotides, sequence variants and fragments thereof, as described herein, may be used to generate hybridization probes or PCR primers for use in such assays. Such probes and primers will be capable of detecting polynucleotide sequences, including genomic sequences that are similar, or complementary to, the peptide polynucleotides described herein.

The invention includes primer pairs for carrying out a PCR to amplify a segment of a polynucleotide of the invention. Each primer of a pair is an oligonucleotide having a length of between 15 and 30 nucleotides such that i) one primer of the pair forms a perfectly matched duplex with one strand of a polynucleotide of the invention and the other primer of the pair form a perfectly match duplex with the complementary strand of the same polynucleotide, and ii) the primers of a pair form such perfectly matched duplexes at sites on the polynucleotide that separated by a distance of between 10 and 2500 nucleotides. Preferably, the annealing temperature of each primer of a pair to its respective complementary sequence is substantially the same.

Hybridization probes derived from polynucleotides of the invention can be used, for example, in performing in situ hybridization on tissue samples, such as fixed or frozen tissue sections prepared on microscopic slides or suspended cells. Briefly, a labeled DNA or RNA probe is allowed to bind its DNA or RNA target sample in the tissue section on a prepared microscopic, under controlled conditions. Generally, dsDNA probes consisting of the DNA of interest cloned into a plasmid or bacteriophage DNA vector are used for this purpose, although ssDNA or ssRNA probes may also be used. Probes are generally oligonucleotides between about 15 and 40 nucleotides in length. Alternatively, the probes can be polynucleotide probes generated by PCR random priming primer extension or in vitro transcription of RNA from plasmids (riboprobes). These latter probes are typically several hundred base pairs in length. The probes can be labeled by any of a number of methods, including fluorescent tags, enzymes or radioactive moieties, according to methods well known in the art. The particular detection method will correspond to the type of label utilized on the probe (e.g., autoradiography, X-ray detection, fluorescent or visual microscopic analysis, as appropriate). The reaction can be further amplified in situ using immunocytochemical techniques directed against the label of the detector molecule used, such antibodies directed to a fluorescein moiety present on a fluorescently labeled probe, or against avidin, or marker enzymes (peroxidase, alkaline phosphatase). Specific labeling and in situ detection methods can be found, for example, in Howard, G. C., Ed., Methods in Nonradioactive Detection, Appleton & Lange, Norwalk, Conn., (1993), herein incorporated by reference. One preferred assay for detecting nucleic acids encoding peptides utilizes the subject polynucleotides, or fragments thereof, as primers in a PCR-based assay. According to the assay, nucleic acids present in a test tissue or cell sample are amplified by polymerase chain reaction (PCR) using two primers consisting of at least 15 nucleotides of a nucleic acid sequence of SEQ ID NOS 6, 7 or 8, or encoding one or more peptides selected from the group consisting of SEQ ID NOs: 11 to 18, including primers derived from variants and/or extensions of such sequences, as described herein. Amplification products are detected in the sample by a method that is appropriate to the particular label used to label the amplification products, according to methods as described in U.S. Pat. No. 4,683,195. For use in PCR detection methods, such as PCR in situ hybridization, PCR primers are selected to be at least 15 nucleotides in length, and preferably between about 15 and 30 nucleotides in length, and are selected from the DNA molecule of interest, according to methods known in the art. While such primers can be selected from within the sequences identified as SEQ ID NOs: 6, 7, or 8, herein, it may also be desirable to select sequences that encompass the longer nucleotide sequences. Preferably, the probes are selected such that the two hybridization sites are separated by between about 10 to 1,000 nucleotides (occasionally up to about 10,000 nucleotides). PCR in situ hybridization of tissue sections and/or cell samples provides a highly sensitive detection method for rare cell types in fixed cell or tissue samples. The PCR in situ hybridization detection method is carried out in accordance with methods that are known in the art, e.g., Nuovo, G. J., PCR IN SITU HYBRIDIZATION: PROTOCOLS AND APPLICATIONS, Raven Press, New York, 1992; U.S. Pat. No. 5,538,871, both of which are incorporated herein by reference.

Briefly, a cell sample (tissue on microscopic slide, pelleted cell suspension) is fixed using a common fixative preparation, such as buffered formalin, formaldehyde or the like. Proteinase or detergent treatment is favored following fixation, to increase cell permeability to reagents. The PCR reaction is carried out in situ by polymerase chain reaction (PCR) using two primers. As discussed above, the primers are designed to selectively amplify one or more of the nucleotide sequences described herein, and particularly sequences described as SEQ ID NOs: 6, 7, or 8. The amplification reaction mixture contains, in addition to the target nucleotide sample and the primers, a thermostable DNA polymerase, such as a polymerase derived from Themus aquaticus (Taq polymerase, U.S. Pat. No. 4,889,818), and a sufficient quantity of the four standard deoxyribonucleotides (dNTPs), one or more of which may be labeled to facilitate detection. The reaction mixture is subjected to several rounds of thermocycling to produce multiple copies (amplification products) of the target nucleotide sequence. Amplification products are then detected in the sample, for example by detecting radioactively labeled amplification products. Hybridization probes and PCR primers may also be selected from the genomic.sequences corresponding to the full-length proteins identified in accordance with the present invention, including promoter, enhancer elements and introns of the gene encoding the naturally occurring peptide. Nucleotide sequences encoding a Carcinoma-related peptide can also be used to construct hybridization probes for mapping the gene which encodes that peptide and for the genetic analysis of individuals. The nucleotide sequences provided herein may be mapped to a chromosome and specific regions of a chromosome using known techniques, such as in situ hybridization, linkage analysis against known chromosomal markers, and hybridization screening with libraries. Briefly, sequences can be mapped to chromosomes by preparing PCR primers (preferably 15-25 bp) from the peptide cDNA. Computer analysis of the 3′ untranslated region is used to rapidly select primers that do not span more than one exon in the genomic DNA, which would complicate the amplification process. Individuals carrying variations of, or mutations in the gene encoding an of the present invention may be detected at the DNA level by a variety of techniques. Nucleic acids used for diagnosis may be obtained from a patient's cells, including, for example, tissue biopsy and autopsy material. Genomic DNA may be used directly for detection or may be amplified enzymatically by using PCR [Saiki, et al. Nature 324:163-166 (1986)] prior to analysis. RNA or cDNA may also be used for the same purpose. As an example, PCR primers complementary to the nucleic acid of the present invention can be used to identify and analyze mutations in the gene of the present invention. Deletions and insertions can be detected by a change in size of the amplified product in comparison to the normal genotype. Point mutations can be identified by hybridizing amplified DNA to radiolabeled RNA of the invention or alternatively, radiolabeled antisense DNA sequences, of the invention. Sequence changes at specific locations may also be revealed by nuclease protection assays, such RNase and S1 protection or the chemical cleavage method [e.g. Cotton, et al., Pvoc. Natl. Acad. Sci. USA 85:4397-4401 (1985)], or by differences in melting temperatures. “Molecular beacons” [Kostrikis L. G. et al., Science 279:1228-1229 (1998)], hairpin-shaped, single-stranded synthetic oligonucleotides containing probe sequences which are complementary to the nucleic acid of the present invention, may also be used to detect point mutations or other sequence changes as well as monitor expression levels of peptide.

Antisense Compounds.

Oligonucleotides of the invention, including pcr primers and antisense compounds, are synthesized by conventional means on a commercially available automated DNA synthesizer, e.g. an Applied Biosystems (Foster City, Calif.) model 380B, 392 or 394 DNA/RNA synthesizer, or like instrument. Preferably, phosphoramidite chemistry is employed, e.g. as disclosed in the following references: Beaucage and Iyer, Tetrahedron, 48: 2223-2311 (1992); Molko et al, U.S. Pat. No. 4,980,460; Koster et al, U.S. Pat. No. 4,725,677; Caruthers et al, U.S. Pat. Nos. 4,415,732; 4,458,066; and 4,973,679; and the like. For therapeutic use, nuclease resistant backbones are preferred. Many types of modified oligonucleotides are available that confer nuclease resistance, e.g. phosphorothioate, phosphorodithioate, phosphoramidate, or the like, described in many references, e.g. phosphorothioates: Stec et al, U.S. Pat. No. 5,151,510; Hirschbein, U.S. Pat. No. 5,166,387; Bergot, U.S. Pat. No. 5,183,885; phosphoramidates: Froehler et al, International application PCT/US90/03138; and for a review of additional applicable chemistries: Uhlmann and Peyman (cited above). Antisense compounds of the invention are selected so that they are complementary to a contiguous subsequence of SEQ ID NO: 8. The length of the antisense oligonucleotides, i.e. such contiguous subsequence, is sufficiently large to ensure that specific binding will take place only at the desired target polynucleotide and not at other fortuitous sites. The upper range of the length is determined by several factors, including the inconvenience and expense of synthesizing and purifying oligomers greater than about 30-40 nucleotides in length, the greater tolerance of longer oligonucleotides for mismatches than shorter oligonucleotides, and the like. Preferably, the antisense oligonucleotides of the invention have lengths in the range of about 15 to 40 nucleotides. More preferably, the oligonucleotide moieties have lengths in the range of about 18 to 25 nucleotides.

### Purification

When peptides of the present invention are expressed in soluble form, for example as a secreted product of transformed yeast or mammalian cells, they can be purified according to standard procedures of the art, including steps of ammonium sulfate precipitation, ion exchange chromatography, gel filtration, electrophoresis, affinity chromatography, and/or the like, e.g. “Enzyme Purification and Related Techniques,” Methods in Enzymology, 22:233-577 (1977), and Scopes, R., Protein Purification: Principles and Practice (Springer-Verlag, New York, 1982) provide guidance in such purifications. Likewise, when peptides of the invention are expressed in insoluble form, for example as aggregates, inclusion bodies, or the like, they can be purified by standard procedures in the art, including separating the inclusion bodies from disrupted host cells by centrifugation, solublizing the inclusion bodies with chaotropic and reducing agents, diluting the solubilized mixture, and lowering the concentration of chaotropic agent and reducing agent so that the peptide takes on a biologically active conformation. The latter procedures are disclosed in the following references, which are incorporated by reference: Winkler et al, Biochemistry, 25: 4041-4045 (1986); Winkler et al, Biotechnology, 3: 992-998 (1985); Koths et al, U.S. Pat. No. 4,569,790; and European patent applications 86306917.5 and 86306353.3.

PEPTIDE can be purified from culture supernatants of mammalian cells transiently transfected or stably transformed by an expression vector carrying an peptide gene. Preferably, peptide is purified from culture supernatants of COS 7 cells transiently transfected by the pcD expression vector. Transfection of COS 7 cells with pcD proceeds as follows: One day prior to transfection, approximately 10⁶ COS 7 monkey cells are seeded onto individual 100 mm plates in Dulbecco's modified Eagle medium (DME) containing 10% fetal calf serum and 2 mM glutamine. To perform the transfection, the medium is aspirated from each plate and replaced with 4 ml of DME containing 50 mM Tris HCl pH 7.4, 400 mg/ml DEAE-Dextran and 50 □g of plasmid DNA. The plates are incubated for four hours at 37° C., then the DNA-containing medium is removed, and the plates are washed twice with 5 ml of serum-free DME. DME is added back to the plates which are then incubated for an additional 3 hrs at 37° C. The plates are washed once with DME, after which DME containing 4% fetal calf serum, 2 mM glutamine, penicillin (100 U/L) and streptomycin (100 □g/L) at standard concentrations is added. The cells are then incubated for 72 hrs at 37° C., after which the growth medium is collected for purification of peptide. Alternatively, transfection can be accomplished by electroporation as described in the examples. Plasmid DNA for the transfections is obtained by growing pcD(SR□), or like expression vector, containing the peptide cDNA insert in E. coli MC1061, described by Casadaban and Cohen, J. Mol. Biol., Vol. 138, pgs. 179-207 (1980), or like organism. The plasmid DNA is isolated from the cultures by standard techniques, e.g. Sambrook et al., Molecular Cloning: A Laboratory Manual, Second Edition (Cold Spring Harbor Laboratory, New York, 1989) or Ausubel et al (1990, cited above).

### EXAMPLE 1

### Monoclonal Antibodies Specific for Carcinoma-Related Peptides

A male Lewis rat is immunized with semi-purified preparations of COS 7-cell expressed peptide. The rat is first immunized with approximately 50 □g of peptide in Freund's Complete Adjuvant, and boosted twice with the same amount of material in Freund's Incomplete Adjuvant. Test bleeds are taken. The animal is given a final boost of 25 □g in phosphate-buffered saline, and four days later the spleen is obtained for fusion.

Approximately 3×10⁸ rat splenocytes are fused with an equal number of P3X63-AG8.653 mouse myeloma cells (available from the ATCC under accession number CRL 1580). 3840 microtiter plate wells are seeded at 5.7×10⁴ parental myeloma cells per well. Standard protocols for the fusion and subsequent culturing of hybrids are followed, e.g. as described by Chretien et al, J. Immunol. Meth., Vol. 117, pgs. 67-81 (1989). 12 days after fusion supernatants are harvested and screened by indirect ELISA on PVC plates coated with COS 7-produced peptide.

### EXAMPLE 2

### Antibodies Specific for a Carcinoma-Related Peptide

50 mg of ovalbumin (OVA) and 50 mg of myoglobulin (MYO) (e.g. available from Sigma) are each dissolved in 10 ml of 0.1 M sodium bicarbonate, and reacted with 1 ml of 0.12 iodoacetamide solution (88 mg of iodoacetamide dissolved in 4 ml 0.1 M sodium bicarbonate) for 1 hour at room temperature in a 15 ml Falcon tube (Falcon Plastics, Oxnard, Calif.), or the like. Each reaction mixture is dialyzed overnight against 4 liters of 0.1 M sodium bicarbonate at 4RC. Separately, 10 mg of carcinoma-related peptide is dissolved in 2 ml of 0.1 M DTT (dithiotheitol) solution (containing 50 mM Tris and 2.5 mM EDTA at pH8) in a 4 ml tube, incubated at 37° C. overnight; and then applied to a GF05 gel-filtration column (1.5×26.5 cm) (LKB, Bromma, Sweden) and eluted with a peptide elution buffer consisting of 0.015 M acetic acid and 0.005 M beta-mercaptoethanol. Three fractions of about 3.5 ml each which contained the reduced peptide are identified by optical density at 206 nm, collected, pooled, frozen in dry ice, and lyophilized overnight. Meanwhile OVA and MYO are recovered from dialysis, and clarified by filtration through 0.45 micrometer filters. OVA and MYO are activated by mixing each with 380 microliters of N-hydroxysuccinimide ester of iodoacetic acid (NHIA) (disclosed by Rector et al., in J. Immunol. Meth., Vol. 24, pg. 321 (1978)) dissolved in tetrahydrofuran (THF) (5 mg/ml); stirring for 30 minutes at room temperature, and dialyzing overnight against 4 liters PBS (1.8 g NaH₂PO₄—H₂O, 7.2 g Na₂HPO₄—H₂O; and 34 g NaCl in 4 liters H₂O). Separately the lyophilized peptide is resuspended in 5 ml of borate reduction buffer (2 g Na₂B₄O₇—10H₂O, 17.4 g NaCl, and 336 mg EDTA-Na₂ in liter H₂O with pH adjusted to 8.5 with concentrated HCl, deoxygenated under nitrogen for 15 minutes, after which 178 mg ascorbate is added). The dialyzed iodoacetylated OVA and MYO are recovered, separately mixed with equal volumes (preferably 2 ml) of borate reduction buffer containing the peptide, and incubated overnight at room temperature. The resulting conjugates are analyzed by SDS-PAGE (12.5% gel). The conjugate containing solution is diluted with PBS to 1 mg/ml, sterile filtered, and aliquotted to convenient volumes (e.g. 500 microliters) for immunizations, and/or stored at 4° C. Polyclonal anti-sera against the MYO conjugate is produced in both rats and rabbits (New Zealand White). The immunization schedule for rabbits is as follows: Initially (week 0) a 10 ml sample of serum is extracted as a control. One week later (week 1) 0.5 ml of peptide-carrier conjugate is mixed with 0.5 ml Freund's Complete Adjuvant and injected I.P. Three weeks later (week 4) a booster is given consisting of 0.5 ml peptide-carrier conjugate mixed with 0.5 ml Freund's Incomplete Adjuvant. The following week (week 5) an additional booster is given, again consisting of 0.5 ml peptide-carrier conjugate mixed with 0.5 ml Freund's Incomplete Adjuvant, followed by yet another identical booster the next week (week 6). On week 7, 20 ml of serum is bled from the animal. After separating out the cellular fraction the serum assayed for positive anti-carcinoma-related peptide titer by ELISA. Rat immunization proceed similarly except that the initial injection consists of 0.15 ml PBS and 0.1 ml peptide-carrier conjugate mixed with 0.75 ml Freund's Complete Adjuvant, boosters consisted of 0.15 ml PBS and 0.1 ml peptide-carrier conjugate mixed with 0.75 ml Freund's Incomplete Adjuvant, and only 2-3 ml of serum is bled from the rat. Again, a positive anti-carcinoma-related peptide reaction is detected by ELISA.

The descriptions of the foregoing embodiments of the invention have been presented for purpose of illustration and description. They are not intended to be exhaustive or to limit the invention to the precise forms disclosed, and obviously many modifications and variations are possible in light of the above teaching. The embodiments were chosen and described in order to best explain the principles of the invention to thereby enable others skilled in the art to best utilize the invention in various embodiments and with various modifications as are suited to the particular use contemplated. It is intended that the scope of the invention be defined by the claims appended hereto.

1. A kit for detecting the presence of carcinoma-related peptides in a biological sample, the kit comprising at least one monoclonal antibody specific for a peptide having an amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

2. A composition of matter comprising a peptide having an amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

3. A nucleic acid, said nucleic acid comprising a nucleotide sequence encoding a polypeptide having an amino acid sequence selected from the group consisting of SEQ ID NOS 11 to 18.

4. The nucleic acid of claim 3, wherein said nucleic acid is operably linked to a promoter.

5. An expression cassette comprising the nucleic acid of claim 4.

6. A host cell comprising the expression cassette of claim 5.

7. A method of making a carcinoma-related peptide, said method comprising:
a) providing a population of host cells comprising a nucleic acid encoding a carcinoma-related peptide of claim 4; and
b) culturing said population of host cells under conditions conducive to the expression of said recombinant nucleic acid;
whereby said peptide is produced within said population of host cells.

8. A method of determining whether a carcinoma-related peptide, or a fragment thereof, is expressed within biological sample, said method comprising the steps of:
a) contacting a biological sample with a detectable polypeptide that selectively binds to a carcinoma-related peptide having an amino acid sequence of any of SEQ ID No 11 to 18; and
b) detecting the presence or absence of binding of said detectable polypeptide to a polypeptide within said sample;
wherein a detection of said hybridization or of said binding indicates that said carcinoma-related peptide is expressed within said sample.

9. An antibody that selectively binds to a peptide having an amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18.

10. A method of binding a carcinoma-related peptide, said method comprising (a) providing a peptide having an amino acid sequence selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, and SEQ ID NO: 18; and (b) bringing said peptide into contact with an antibody that binds said peptide.

11. A method of making a carcinoma-related peptide, said method comprising:
a) providing a population of host cells comprising a nucleic acid encoding a carcinoma-related peptide of claim 5; and
b) culturing said population of host cells under conditions conducive to the expression of said recombinant nucleic acid;
whereby said peptide is produced within said population of host cells.
